(19) United States
(12) Patent Application Publication
(10) Pub. No.: US 2026/0235584 A1
(43) Pub. Date: Aug. 13, 2026

(54) DEVICES, SYSTEMS, AND METHODS FOR OPTICAL ANALYSIS OF BIOLOGICAL SAMPLES

(71) Applicant: VITAL BIOSCIENCES INC., Oakville (CA)

(72) Inventors: Akhil Rajagopal KOZHIPURAM, Brampton (CA); John REEP, San Diego, CA (US); Abdolreza MOVAGHAR, San Diego, CA (US); Mark Ronald BREMER, Cambridge (CA); Aaron CAMPBELL, Mount Hope (CA); Todd CAMPBELL; Bruce ECKARD, San Diego, CA (US); Erden ERTORER, Niagara Falls (CA); Christopher FETTERS, Street, MD (US); Farnoud KAZEMZADEH, Waterloo (CA); Iman KHODADADZADEH, San Carlos, CA (US); Mounir KOUSSA, Oakville (CA); Ross MACLEOD, Wilmington, MA (US); Srinivas NADELLA, San Carlos (CA); Claire Grace NOEL, King City (CA); Paul Said Ehrlich PEREZ, Toronto (CA); Ryan ROOT, El Cajon, CA (US); Sergey SANDERS, Cambridge (CA)

(73) Assignee: VITAL BIOSCIENCES INC., Oakville, ON (CA)

(21) Appl. No.: 19/648,418

(22) Filed: Apr. 15, 2026

Related U.S. Application Data
(63) Continuation of application No. PCT/IB2024/060227, filed on Oct. 17, 2024.
(60) Provisional application No. 63/591,603, filed on Oct. 19, 2023.

(51) Int. Cl. G01N 33/53; G01N 35/00; G01N 35/10

(52) U.S. Cl. CPC G01N 33/5302; G01N 35/00693; G01N 35/0099; G01N 35/10; G01N 2035/00346; G01N 2035/00465

(57) ABSTRACT

A system for optical analysis of biological samples is disclosed. The system may be used as a point of care system and include one or more analytical modules, each configured for receiving a corresponding microfluidic disc and performing optical analysis of a corresponding biological sample. The system may include a housing, a chassis and gantry unit, a drawer, a gripper, a pipetting device, a rocker, a capping/decapping device, a plurality of analytical modules, a detection unit, a thermal management unit, a control unit, or any combination thereof.

### CROSS-REFERENCE TO RELATED APPLICATIONS

This application claims the benefit of U.S. Provisional Patent Application No. 63/591,603 filed Oct. 19, 2023, entitled “Devices, Systems and Methods for Optical Analysis of Biological Samples”, which is incorporated by reference herein in its entirety.

### TECHNICAL FIELD

The present disclosure relates to devices, systems and methods that allow running reactions and analyzation of biological samples in microfluidic devices.

### BACKGROUND

Currently, 70% of all medical decisions rely on lab-based diagnostics but today, the diagnostic process is disjointed from how care is delivered. The primary care system requires patients to travel to external phlebotomy sites to draw blood, which is sent to labs via courier, and processed overnight. This means that lab results reach health care professionals long after the patient has left. This friction in care delivery and disease management leads to tremendous waste in the healthcare system:
a. Patients often delay getting lab tests or fail to adhere to lab testing, or subsequent care recommendations;
b. The gap in the diagnostic process leads to missed tests, missed diagnosis, a lack of intervention, and ultimately poor outcomes; and
c. Health care professionals waste time tracing lab orders to patient encounter notes. When intervention is needed, additional time is wasted reaching out to patients and driving subsequent steps in the patient's care pathway.

These problems are even more acute when caring for rural populations or patients belonging to groups facing adverse social-determinants of health, where there are many challenges in ensuring successful follow ups from an initial patient encounter.

Several companies have built point-of-care instruments to bridge this divide. However, these instruments are limited to single types of tests and fail to completely meet the workflow needs of primary care providers for a single system that produces simple, comprehensive, and fast test results. A product to meet these needs is currently under development. It achieves this through a highly automated workflow, enabled through the use of microfluidics.

Moreover, different reactions may last over very different time periods. For instance, one endpoint assay may take less than a few minutes to run and the other may take more than ten minutes to run. In many conventional settings, one has to synchronize all the chemistries to finish at the same time and then perform the measurement. Further, different assays may rely on different wavelengths for illumination or detection. For a large number of assays, this may take a significant amount of time and resources, and in some cases may not be practical.

Accordingly, there remains a need for improved devices, systems and methods for performing a large number of reactions and analyzation in parallel using microfluidics technology, allowing for user-friendly and point-of-care deployment of such technology.

### SUMMARY

In one embodiment, there is a system including one or more analytical modules, each configured for receiving a corresponding microfluidic disc and performing optical analysis of a corresponding biological sample. In some embodiments, the one or more analytical modules consist of a single analytical module. In some embodiments, the one or more analytical modules comprise a plurality of analytical modules. In some embodiments, analytical modules in the plurality of analytical modules are capable of performing optical analyses independently of each other or concurrently with each other. In some embodiments, the plurality of analytical modules are capable of performing a full suite of tests.

In some embodiments, the one or more analytical modules include a first analytical module configured for receiving a first microfluidic disc and measuring one or more hematological parameters, a second analytical module configured for receiving a second microfluidic disc and performing an immunoassay, a third analytical module configured for receiving a third microfluidic disc and performing a clinical chemistry analysis, or any combination thereof.

In some embodiments, the system includes a gripper configured for transporting the corresponding microfluidic disc to or from each of the one or more analytical modules, a holder configured for holding one or more sample tubes, a rocker configured for mixing or homogenizing one or more biological samples in the one or more sample tubes, a capping/decapping device configured for decapping and/or recapping a cap from a sample tube in the one or more sample tubes, a drawer configured for receiving consumables, a pipetting device configured for pipetting the corresponding biological sample to the corresponding microfluidic disc, a detection unit configured for one or more of detecting any microfluidic failure on the microfluidic disc in each of the plurality of analytical modules, detecting a position of the gripper, the holder, the rocker, the capping/decapping device, the drawer, or any combination thereof, thereby providing information for auto-calibration of the system and performing one or more assay, a thermal management unit configured for maintaining the system at a predetermined temperature range, or any combination thereof. In some embodiments, the detection unit comprises one or more image cameras.

In some embodiments, the system includes a housing comprising a door and a user interface, a chassis, and a gantry. In some embodiments, the system includes a controlling unit for operating the gripper, the holder, the rocker, the capping/decapping device, the drawer, the pipetting device, the plurality of analytical modules, the detection unit, the thermal management unit, or any combination thereof. In some embodiments, the one or more analytical modules comprise at least three analytical modules powered by a single and unified architecture and capable of being performed in a modular and streamlined platform. In some embodiments, the corresponding biological sample is aliquoted into the corresponding microfluidic disc for the optical analysis.

In some embodiments, the system is configured to accept a single consumable kit per sample. In some embodiments, the one or more analytical modules are capable of providing a plurality of biomarkers results. In some embodiments, the one or more analytical modules are capable of providing 2, 3, 4, 5, 6, 7, 8, 9, 10, 11, 12, 13, 14, 15, 16, 17, 18, 19, 20, 30, 40, 50, 60, 70, 80, 90, 100 or more than 100 biomarkers results. In some embodiments, the one or more analytical modules are capable of providing more than 5 biomarkers results.

In some embodiments, optical analysis of the corresponding biological sample is performed using a single workflow with user-selectable reporting. In some embodiments, the system is automated. In some embodiments, the system is configured to accept one or more sample tubes, homogenize one or more biological samples contained in the one or more sample tubes, and analyze the homogenized one or more biological samples without any user intervention. In some embodiments, the system is configured to assess a quality of samples, reagents, and/or protocols. In some embodiments, the system is configured to transmit information about a quality of samples, reagents, and/or protocols using telemetry information.

### BRIEF DESCRIPTION OF THE DRAWINGS

The accompanying drawings, which are incorporated into and constitute a part of this specification, illustrate one or more exemplary embodiments of the present disclosure and, together with the Summary and Detailed Description, serve to explain the principles and implementations of exemplary embodiments of the invention. The accompanying drawings are not necessarily to scale. The specific design features of the present invention as disclosed herein, including, for example, specific dimensions, orientations, locations, and shapes, will be determined in part by the particular intended application and use environment. In addition, the components illustrated in the figures are combinable in any useful number and combination. In the drawings, like reference numerals indicate like elements throughout.

In the drawings:

FIG. 1A is a perspective view schematically illustrating a system in accordance with some exemplary embodiments of the present disclosure;

FIG. 1B is a perspective view schematically illustrating an interior of the system of FIG. 1A in accordance with some exemplary embodiments of the present disclosure;

FIG. 1C is another perspective view schematically illustrating an interior of the system of FIG. 1A in accordance with some exemplary embodiments of the present disclosure;

FIG. 1D is a top view schematically illustrating an interior of the system of FIG. 1A in accordance with some exemplary embodiments of the present disclosure;

FIG. 1E is a perspective view schematically illustrating some components of the system of FIG. 1A in accordance with some exemplary embodiments of the present disclosure;

FIG. 1F is a perspective view schematically illustrating a portion of FIG. 1E;

FIG. 1G is an enlarged view illustrating a portion of FIG. 1F;

FIG. 2A is a perspective view schematically illustrating an exemplary gripper in accordance with some exemplary embodiments of the present disclosure;

FIG. 2B is an exploded view schematically illustrating the gripper of FIG. 2A in accordance with some exemplary embodiments of the present disclosure;

FIG. 2C is a cross-sectional view schematically illustrating the gripper of FIG. 2A in a first state in accordance with some exemplary embodiments of the present disclosure;

FIG. 2D is a cross-sectional view schematically illustrating the gripper of FIG. 2A in a second state in accordance with some exemplary embodiments of the present disclosure;

FIG. 2E is a cross-sectional view schematically illustrating the gripper of FIG. 2A in a third state in accordance with some exemplary embodiments of the present disclosure;

FIG. 3A is a perspective view schematically illustrating an exemplary drawer, along with some exemplary consumable packs and other components, in accordance with some exemplary embodiments of the present disclosure;

FIG. 3B is a perspective view schematically illustrating the exemplary drawer of FIG. 3A without the exemplary consumable packs in accordance with some exemplary embodiments of the present disclosure;

FIG. 3C is an exploded view schematically illustrating the exemplary drawer of FIG. 3A with the exemplary consumable packs and some other components in accordance with some exemplary embodiments of the present disclosure;

FIG. 3D is an exploded view schematically illustrating some components of FIG. 3A in accordance with some exemplary embodiments of the present disclosure;

FIG. 3E is a perspective view schematically illustrating some components of FIG. 3A in accordance with some exemplary embodiments of the present disclosure;

FIG. 3F is a perspective view schematically illustrating some components of FIG. 3A in accordance with some exemplary embodiments of the present disclosure;

FIG. 3G is a perspective view schematically illustrating some components of FIG. 3A in accordance with some exemplary embodiments of the present disclosure;

FIG. 3H is another perspective view schematically illustrating some components of the exemplary drawer of FIG. 3A in accordance with some exemplary embodiments of the present disclosure;

FIGS. 3I, 3J, 3K and 3L are perspective or side views schematically illustrating an exemplary use of the exemplary drawer of FIG. 3A in accordance with some exemplary embodiments of the present disclosure;

FIGS. 3M and 3N are partially cut-out views schematically illustrating some components of the exemplary drawer of FIG. 3A in accordance with some exemplary embodiments of the present disclosure;

FIGS. 3O, 3P, 3Q and 3R are top views schematically illustrating some components of the exemplary drawer of FIG. 3A in accordance with some exemplary embodiments of the present disclosure;

FIG. 4A is a perspective view schematically illustrating an exemplary rocker in a first state, along with some other components, in accordance with some exemplary embodiments of the present disclosure;

FIG. 4B is a perspective view schematically illustrating the exemplary rocker of FIG. 4A in a second state, along with some other components, in accordance with some exemplary embodiments of the present disclosure;

FIG. 4C is a side view schematically illustrating the exemplary rocker of FIG. 4A in the first state, along with some other components, in accordance with some exemplary embodiments of the present disclosure;

FIG. 4D is a side view schematically illustrating the exemplary rocker of FIG. 5B in the second state, along with some other components, in accordance with some exemplary embodiments of the present disclosure;

FIG. 4E is a perspective view schematically illustrating the exemplary rocker of FIG. 4A, along with some other components, in accordance with some exemplary embodiments of the present disclosure;

FIG. 4F is a perspective view schematically illustrating the exemplary rocker of FIG. 4A, where an exemplary tube rack is omitted for clarity, in accordance with some exemplary embodiments of the present disclosure;

FIG. 4G is an exploded view schematically illustrating the exemplary rocker of FIG. 4F in accordance with some exemplary embodiments of the present disclosure;

FIG. 4H is another exploded view schematically illustrating the exemplary rocker of FIG. 4F in accordance with some exemplary embodiments of the present disclosure;

FIG. 4I is a perspective view schematically illustrating the exemplary rocker of FIG. 4A, where a cam surface does not abut against an external component, in accordance with some exemplary embodiments of the present disclosure;

FIG. 4J is a close-up view schematically illustrating the cam surface and the external component of FIG. 4I in accordance with some exemplary embodiments of the present disclosure;

FIG. 4K is a perspective view schematically illustrating the exemplary rocker of FIG. 4A, where a cam surface abuts against an external component, in accordance with some exemplary embodiments of the present disclosure;

FIG. 4L is a close-up view schematically illustrating the cam surface and the external component of FIG. 4K in accordance with some exemplary embodiments of the present disclosure;

FIG. 4M is a sectional view schematically illustrating the position of a tube retaining member of FIG. 4I in accordance with some exemplary embodiments of the present disclosure;

FIG. 4N is a sectional view schematically illustrating the position of a tube retaining member of FIG. 4J in accordance with some exemplary embodiments of the present disclosure;

FIG. 5A is a perspective view schematically illustrating an exemplary capping/decapping device in accordance with some exemplary embodiments of the present disclosure;

FIG. 5B is another perspective view schematically illustrating the exemplary capping/decapping device of FIG. 5A in accordance with some exemplary embodiments of the present disclosure;

FIG. 5C is an exploded view schematically illustrating the exemplary capping/decapping device of FIG. 5A in accordance with some exemplary embodiments of the present disclosure;

FIG. 5D is an enlarged view schematically illustrating a portion of the exemplary capping/decapping device of FIG. 5C.

FIG. 5E is a cross-sectional cutout view schematically illustrating the exemplary capping/decapping device of FIG. 5A in accordance with some exemplary embodiments of the present disclosure;

FIG. 5F is a cross-sectional cutout view schematically illustrating the exemplary capping/decapping device of FIG. 5A, along with a sample tube, in accordance with some exemplary embodiments of the present disclosure;

FIG. 5G is a perspective view schematically illustrating the exemplary capping/decapping device of FIG. 5A positioned in a housing in accordance with some exemplary embodiments of the present disclosure;

FIGS. 5H and 5I are perspective views schematically illustrating an exemplary process of sliding one or more caps into or out of the exemplary capping/decapping device of FIG. 5A in accordance with some exemplary embodiments of the present disclosure;

FIG. 6 is a perspective view schematically illustrating an exemplary detection unit, along with some other components, in accordance with some exemplary embodiments of the present disclosure; and

FIGS. 7A, 7B and 7C are perspective views schematically illustrating an exemplary thermal management unit, along with some other components, in accordance with some exemplary embodiments of the present disclosure.

### DETAILED DESCRIPTION

The present disclosure provides devices, systems, and methods for running reactions in microfluidic devices for hematology analysis, immunoassay analysis, clinical chemistry analysis, or any combination thereof. In some embodiments, the systems are capable of running a full suite of tests and can be used as a point-of-care system.

### Exemplary System

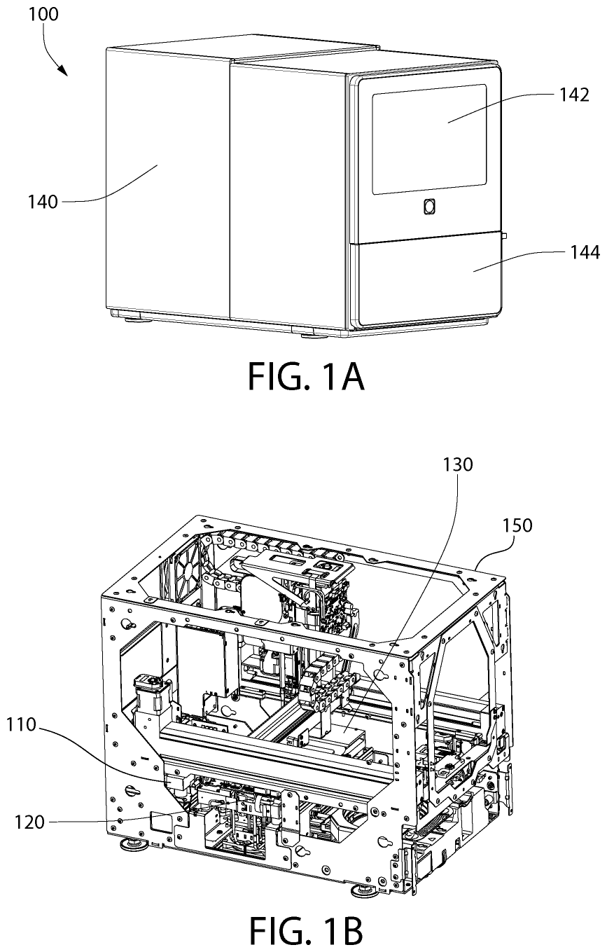

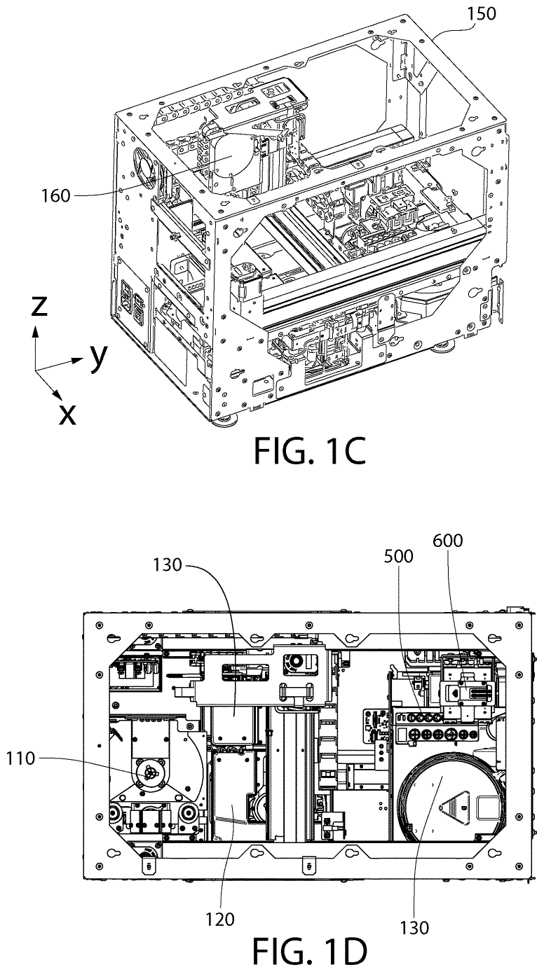

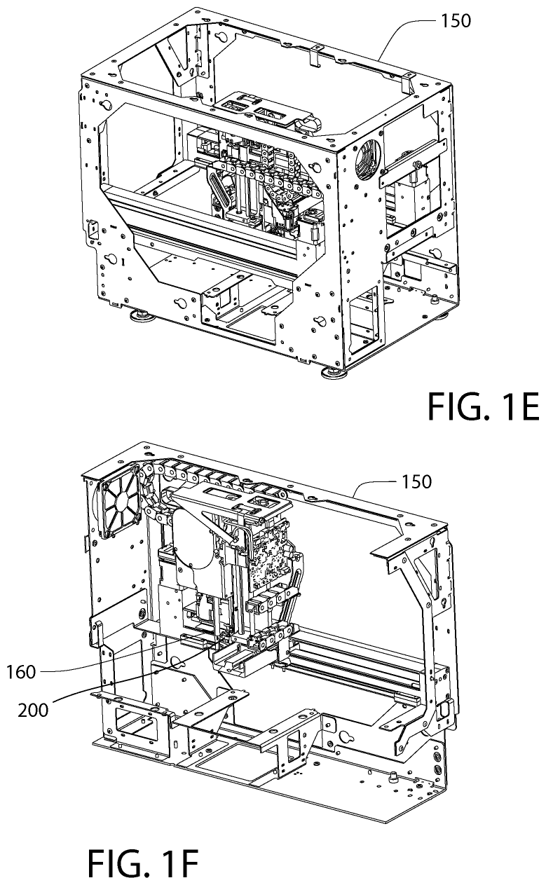

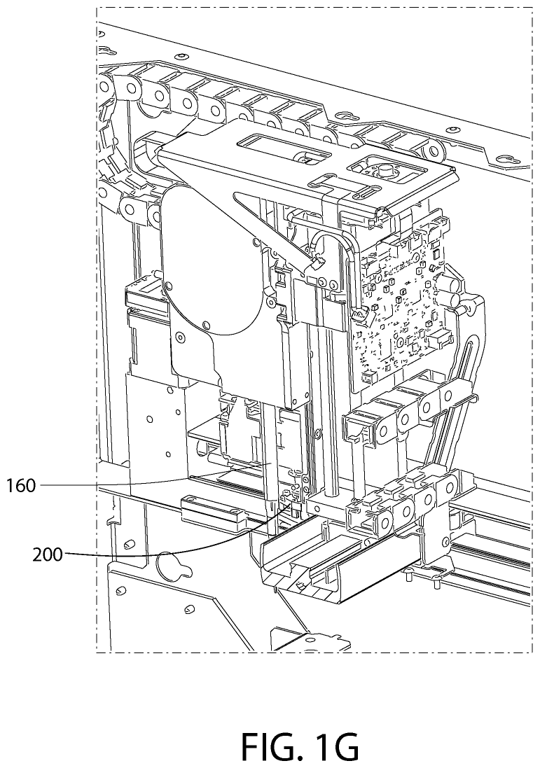

Referring to FIGS. 1A-1D, there is shown an exemplary system 100 in accordance with some embodiments of the present disclosure. The system 100 is configured for running reactions and analyzation of biological samples in microfluidic devices, and may be used for hematology analysis, immunoassay analysis, clinical chemistry analysis, or any combination thereof. In some embodiments, the system 100 is configured for running a full suite of tests. The system 100 may be used as a point-of-care system. The system 100 includes one or more analytical modules, such as a first analytical module 110, a second analytical module 120, and/or a third analytical module 130. Each analytical module is configured for receiving a microfluidic disc and analyzing a biological sample, e.g., for performing hematology analysis, immunoassay analysis, clinical chemistry analysis or other analyses. In some embodiments, the system 100 includes a plurality of analytical modules, each capable of performing an analysis independently or concurrently with the other(s). As a non-limiting example, it is illustrated that the system 100 includes three analytical modules, e.g., the first analytical module 110, the second analytical module 120 and the third analytical module 130, one for hematology analysis (e.g., performing a complete blood count test), one for immunoassay analysis and one for clinical chemistry analysis. In some embodiments, the first analytical module 110 is configured to receive a first microfluidic disc and perform hematology analysis.

The second analytical module 120 is configured to receive a second microfluidic disc and perform immunoassay analysis. The third analytical module 130 is configured to receive a third microfluidic disc and perform clinical chemistry analysis. Examples of such microfluidic discs and analytical modules are disclosed in U.S. Provisional Patent Application No. 63/489,667, filed Mar. 10, 2023, U.S. Provisional Patent Application No. 63/489,677, filed Mar. 10, 2023, U.S. Provisional Patent Application No. 63/489,681, filed Mar. 10, 2023, U.S. Provisional Patent Application No. 63/514,965, filed Jul. 21, 2023, U.S. Provisional Patent Application No. 63/514,973, filed Jul. 21, 2023, and U.S. Provisional Patent Application No. 63/514,978, filed Jul. 21, 2023, the content of each application is hereby incorporated by reference in its entirety.

In some embodiments, the system 100 includes one or more other components, modules, devices, or mechanisms, such as those disclosed herein. For instance, in some embodiments, the system 100 includes a chassis and gantry unit 150, a pipetting device 160, a gripper 200, a drawer 300, a sliding mechanism 400, a rocker 500, a tube holder 503, a capping/decapping device 600, a detection unit 700, a thermal management unit 800, or any combination thereof. The one or more analytical modules, pipetting device, drawer, gripper, holder, rocker, capping/decapping device detection unit, or any combination thereof may be accommodated in a housing 140. In some embodiments, the system 100 also includes a controlling unit for operating the gripper, the holder, the rocker, the capping/decapping device, the drawer, the pipetting device, one or more analytical modules, the detection unit, the thermal management unit 800, or any combination thereof. The controlling unit may include one or more processors and memory storing instructions that, when executed by the one or more processors, cause the system to perform one or more analyses. The control unit may be in the form of one or more printed circuit boards and may be accommodated within the housing or disposed outside of the housing.

The housing 140 may include a user interface 142 to enable interaction between a user and the system 100. For instance, the user interface 142 may be used for receiving an instruction from a user and/or displaying information related to an analysis. The housing 140 may also include a door 144 to allow at least a portion of the drawer 300 to be moved into or out of the housing for placement or retrieval of samples and/or other materials. The drawer 300 may be configured for receiving consumables (e.g., microfluidic disc pack, reagent pack, pipette tip pack). Examples of consumable packs are disclosed in U.S. Provisional Patent Application No. 63/498,228, filed Apr. 25, 2023, the content of the application is hereby incorporated by reference in its entirety.

The gripper 200 may be configured for transporting a microfluidic disc to or from an analytic module. The tube holder 503 may be configured for holding one or more sample tubes. The rocker 500 may be configured for mixing the sample in the one or more sample tubes. The capping/decapping device 600 may be configured for decapping and/or recapping the one or more sample tubes. The pipetting device 160 may be configured for pipetting a sample from a sample tube to a microfluidic disc on an analytic module.

The chassis and gantry unit 150 may be configured for facilitating movement of some parts in one or more directions. For instance, the chassis and gantry unit 150 may be configured to allow the pipetting device 160 and the gripper 200 to move in three directions, e.g., in the x-, y-and z-directions in FIG. 1C. Additionally, or optionally, the chassis and gantry unit 150 may also be configured to allow at least a portion of the drawer 300 to move (e.g., slide) into and out of the housing 140. Additionally, or optionally, the chassis and gantry unit 150 may further be configured to allow at least a portion of the tube holder 503 to move into and out of the housing 140.

The pipetting device 160 and the gripper 200 may be connected to each other and operatively movable in one or more directions jointly. The pipetting device 160 and the gripper 200 may also be operatively movable in one or more directions independently. For instance, in some embodiments, the z-axis of the pipetting device 160 and the z-axis of the gripper 200 may be offset from each other in the x-direction, the y-direction, or both of the x-and y-directions as illustrated in FIGS. 1E-1G. In some such embodiments, the pipetting device 160 and the gripper 200 may be operatively movable in the z-direction independently of each other.

In some embodiments, the system 100 is capable of running a plurality of different methods of analysis, e.g., performing analyses using a plurality of different analytical modules. In some embodiments, the system 100 is capable of running 2, 3, 4, 5 or more than 5 different methods of analysis. In some embodiments, the system 100 is powered by a single and unified architecture, and/or is configured to enable a modular and streamlined platform. In some embodiments, the system 100 is capable of running 3 different methods of analysis in a modular and streamlined platform and powered by a single and unified architecture.

In some embodiments, the system 100 is configured to aliquot the sample into the appropriate method of analysis.

In some embodiments, the system 100 is configured to accept one or more consumable kits per sample. For instance, in some embodiments, the system 100 is capable of accepting a single consumable kit per sample or accepting two or more consumable kits per sample.

In some embodiments, the system 100 is capable of providing one or more biomarkers results. In some embodiments, the system 100 is capable of providing a plurality of biomarkers results, including but not limited to 2, 3, 4, 5, 6, 7, 8, 9, 10, or more than 10 biomarkers results. In some embodiments, the system 100 is capable of providing more than 5 biomarkers results. In some embodiments, the system 100 is capable of providing at least some biomarkers results simultaneously.

In some embodiments, the system 100 is configured to perform an assessment of a biological sample using a single workflow with user-selectable reporting.

In some embodiments, one or more processes are automated. For instance, in some embodiments, the system 100 is automated such that it can accept one or more sample tubes, homogenize the sample(s) in the one or more sample tubes and make the homogenized sample available for assessment without any user intervention.

In some embodiments, the system 100 is capable of assessing the quality of samples, reagents, and protocol using telemetry information, which may include data measured by any sensor or camera disclosed herein or data received via a telecommunication.

The system 100 may be initiated by a user, for instance, by scanning their badge or using the user interface 142. In response, the system 100 will open the door to allow the user to load the sample tube(s) and consumables (e.g., microfluidic disc pack, reagent pack, pipette tip pack) to the system 100 (e.g., the drawer, the tube holder). Then, the user may simply command the system 100 e.g., via the user interface 142, to start the test protocol. In response, the drawer 300 will go into the system 100 and the door 144 will close. The gripper 200 will pick up a disc and load it onto a corresponding analytical module. If there is more than one disc, the gripper 200 will pick up another disc and load it to another analytical module. While the gripper 200 is picking up and loading the disc(s), the rocker 500 may begin rocking the sample tube(s) to mix the sample. After mixing, the sample tube(s) will be moved into the capping/decapping device 600, and the capping/decapping device 600 will decap the sample tube(s). The pipetting device 160 will pick up a pipette tip, collect a sample from the decapped sample tube and pipette it into a corresponding microfluidic disc on a corresponding analytic module. The corresponding analytic module will then perform the analysis in accordance with the user input, the barcode on the sample tube(s), the barcode on the consumables, or any combination thereof. Once the analysis is completed, the capping/decapping device 600 may recap the sample tube(s). The gripper 200 may pick up the disc(s) and transport it back to the consumables in the drawer 300. The door 144 may open and the drawer 300 and the tube holder 503 may move out of the system 100 to allow the user to retrieve the sample tube(s) and consumables.

The system 100 may be used as a point-of-care system. For instance, it may be placed at a primary care physician's office or any other suitable locations. Upon arrival, a patient may give a sample (e.g., blood), and a user (e.g., a physician's assistant, a laboratory technician or personnel who is allowed to operate the system) may take the sample and use the system 100 to analyze the sample (e.g., to run a full suite of tests). By the time the patient is ready to see the physician, all the results are available and the physician is able to review those results with the patient in the same visit.

### Exemplary Gripper

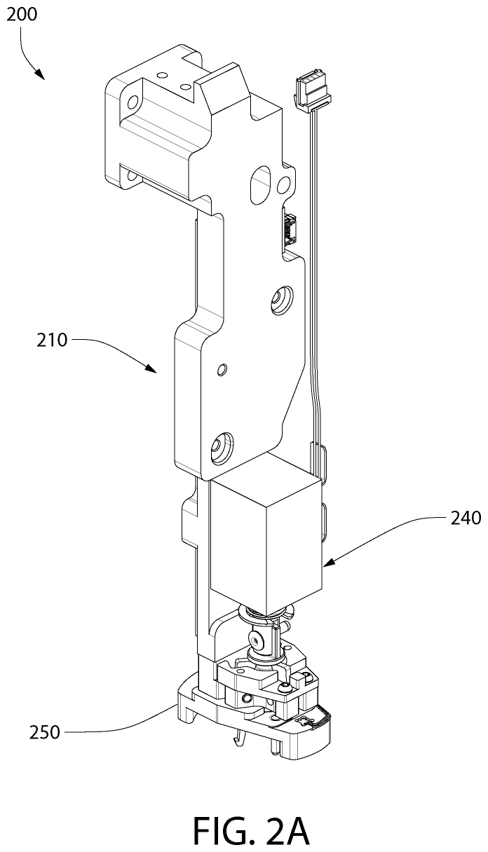

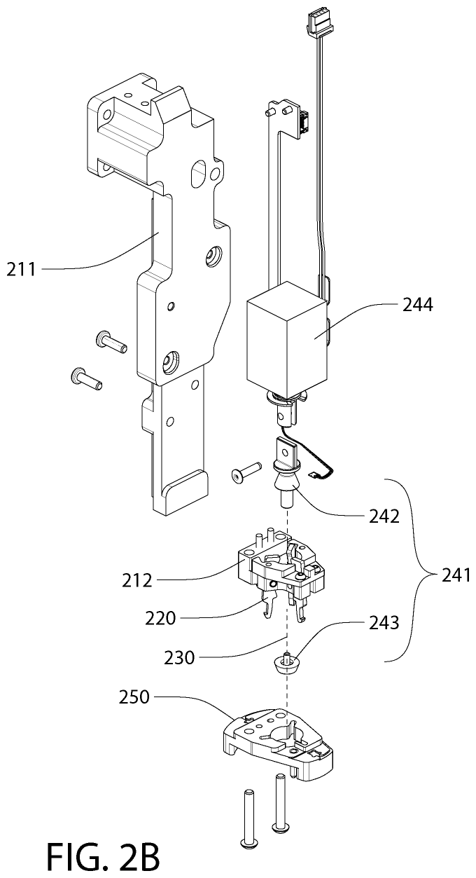

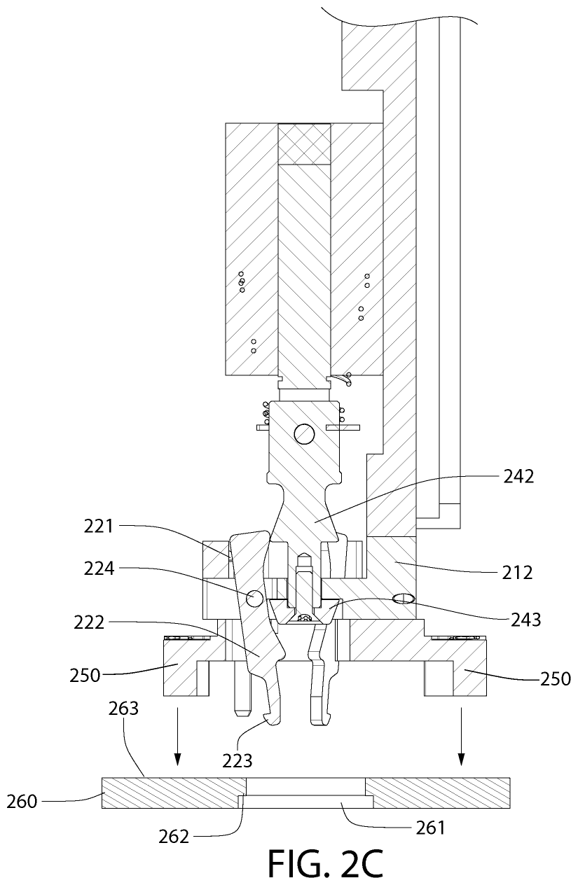

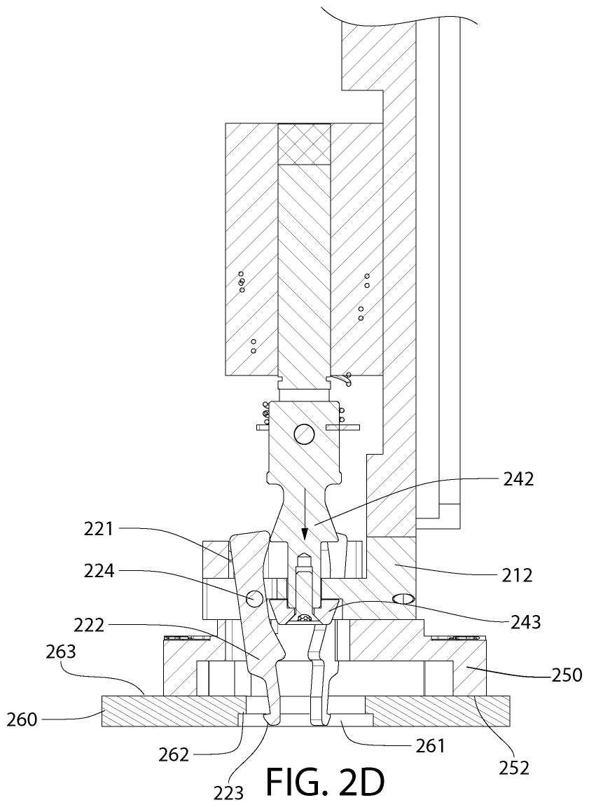

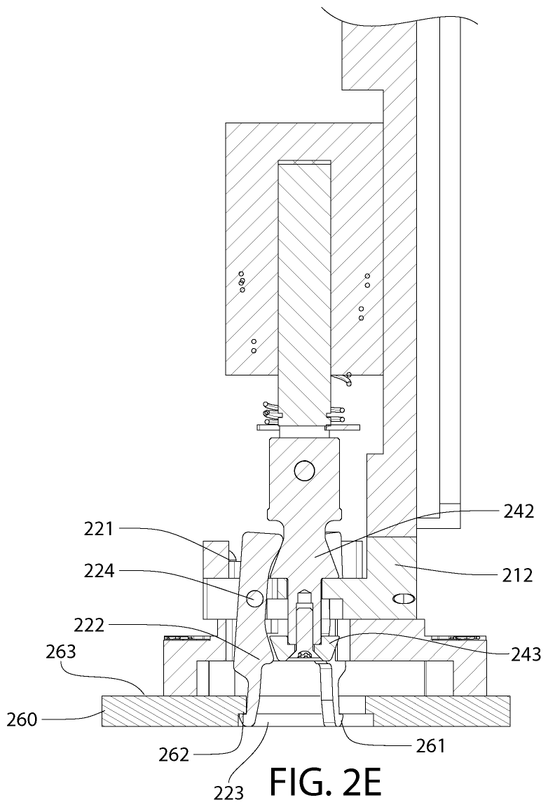

Referring to FIGS. 2A-2E, there is shown an exemplary gripper 200 in accordance with some embodiments of the present disclosure. The gripper 200 is configured for picking up and/or transporting an object, such as a disc 260. The disc 260 may have an aperture 261, a step 262 along at least a portion of a perimeter of the aperture 261, and a disc surface 263. Examples of such a disc are disclosed in U.S. Provisional Patent Application No. 63/489,667, filed Mar. 10, 2023; U.S. Provisional Patent Application No. 63/489,677, filed Mar. 10, 2023; and U.S. Provisional Patent Application No. 63/489,681, filed Mar. 10, 2023, the content of each application is hereby incorporated by reference in its entirety. Compared to conventional grippers, the gripper 200 is more reliable, more stable, and more effective, in particular, in gripping and/or transporting a thin disc.

The gripper 200 generally includes a staging unit 210, a plurality of fingers 220, and a pushing unit 240. The staging unit 210 may be connected to the chassis and gantry unit 150 and operably movable in one or more of the x-, y- and z-directions in the system 100. The staging unit 210 may include one or more components. For instance, in some embodiments, the staging unit 210 includes a base 211 and a pivot plate 212 connected to (e.g., mounted on) the base 211. The base 211 may be configured for connecting the gripper 200 to the chassis and gantry unit 150 of the system 100 so that the gripper 200 can be used for transferring a microfluidic disc to or from an analytic module in the system 100. However, the present disclosure is not limited thereto. The base 211 may be configured for connecting the gripper 200 to any other structures or systems so that the gripper 200 may be used as a standalone device or in other systems that require transferring of objects.

Fingers in the plurality of fingers 220 are disposed circumferentially around a gripping axis 230, with each respective finger in the plurality of fingers 220 pivotally connected to the staging unit 210 at its middle portion. For instance, in the illustrated embodiment, each respective finger 220 is pivotally connected to the pivot plate 212 of the staging unit 210 at a corresponding pivot point 224 and is thus into a first finger portion 221 and a second finger portion 222. In some embodiments, the second finger portion 222 of the respective finger 220 has a gripping part 223. The gripping part 223 can be formed at any suitable locations along the second finger portion 222 and can have any suitable shape and/or size. In some embodiments, the gripping part 223 is formed at an end of the second finger portion 222 of the respective finger 220. In some embodiments, the gripping part 223 includes a hook. In some embodiments, the hook is oriented to face radially outward relative to the gripping axis 230.

The plurality of fingers 220 can include any suitable number of fingers, including but not limited to two, three, four, five, or more than five fingers. Fingers in the plurality of fingers 220 can be, but do not necessarily have to be, identical or symmetric to each other, and can be, but do not necessarily have to be, evenly spaced around the gripping axis 230. As a non-limiting example, FIG. 2B illustrates three substantially identical fingers that are spaced substantially evenly around the gripping axis 230.

The pushing unit 240 is connected to the staging unit 210 (e.g., connected to the base 211 of the staging unit) and configured to operate the fingers 220 to grip or release the object. For instance, in some embodiments, the pushing unit 240 includes a pushing member 241 that is generally aligned with the gripping axis 230. The pushing member 241 is operably movable relative to the staging unit 210 along the gripping axis 230, and accordingly, operably movable relative to the fingers 220 along the gripping axis 230 since the fingers 220 are mounted on the pivot plate 212 of the staging unit 210. As it moves along the gripping axis 230, the pushing member 241 selectively abuts outwardly against the first finger portion 221 or second finger portion 222 of each respective finger 220, and consequently causes the gripping part 223 of each respective finger 220 to move inwardly or outwardly for gripping or releasing the object (e.g., the disc 260). In some embodiments, the pushing member 241 functions as a cam surface.

The pushing member 241 may be made of multiple parts or segments. For instance, in some embodiments, the pushing member 241 includes a first pushing portion 242 and a second pushing portion 243. When the pushing member 241 is at a first pushing position, such as that illustrated in FIG. 2C, the first pushing portion 242 abuts against the first finger portion 221 of each respective finger 220 radially outward relative to the gripping axis 230. This causes each respective finger 220 to rotate around the corresponding pivot point 224, resulting in the gripping part 223 of each respective finger 220 to move radially inward relative to the gripping axis 230. At this state, the gripping parts 223 of the plurality of fingers 220 are capable of passing through the aperture 261 of the disc 260. For instance, in some embodiments, the staging unit 210 is operably movable in a direction that is substantially parallel to the gripping axis 230. By moving the staging unit 210 and/or the disc 260 toward each other, the gripping parts 223 of the plurality of fingers 220 can be inserted into the aperture 261 of the disc 260 as illustrated in FIG. 2D. Similarly, at this state, by moving the staging unit 210 and/or the disc 260 away from each other, the gripping parts 223 of the plurality of fingers 220 can be pulled out of the aperture 261 of the disc 260.

When the gripping parts 223 of the plurality of fingers 220 have been inserted into the aperture 261 of the disc 260 and picking up the disc 260 is desired, the pushing member 241 can be moved to a second pushing position, such as that illustrated in FIG. 2E. At this second pushing position, the second pushing portion 243 abuts against the second finger portion 222 of each respective finger 220 radially outward relative to the gripping axis 230. This causes each respective finger 220 to rotate around the corresponding pivot point in an opposite direction, resulting in the gripping part 223 of each respective finger 220 to move radially outward relative to the gripping axis 230. At this state, the gripping parts 223 of the plurality of fingers 220 are engaged with the step 262 of the disc 260 and thus grip the disc 260. The disc 260 can then be transported to a desired place (e.g., an analytic module), for instance, by moving the stage unit 210 via the chassis and gantry unit 150.

The first pushing portion 242 and the second pushing portion 243 can take any suitable shapes and sizes. In some embodiments, at least one of the first pushing portion 242 and the second pushing portion 243 is substantially a truncated cone. In some embodiments, each of the first pushing portion 242 and the second pushing portion 243 is substantially a truncated cone, with their bases facing each other. In some embodiments, the second pushing portion 243 is smaller than the first pushing portion 242. In some embodiments, the first pushing portion 242 and second pushing portion 243 are spaced apart from each other along the gripping axis 230. In some such embodiments, a portion of the pivot plate 212 is disposed between the first pushing portion 242 and second pushing portion 243, and thus prevents the pushing member 241 from removing completely out of the interior space collectively formed by the plurality of fingers 220.

In some embodiments, the pushing unit 240 includes a driving member 244 to move the pushing member 241. In some embodiments, the driving member 244 includes a solenoid having a housing connected to the staging unit 210 and a coil of wire to operate the pushing member 241. In some embodiments, the driving member 244 includes a stepper motor with lead screw(s) or any other suitable linear actuators to the pushing member 241.

The gripper 200 may include additional, optional or alternative components. For instance, in some embodiments, the gripper 200 includes a datum unit 250 to help stabilize the disc 260 when the disc 260 is gripped by the plurality of fingers 220. The datum unit 250 may be connected to the staging unit 210, e.g., the pivot plate 212 of the staging unit 210. When the disc 260 is gripped by the plurality of fingers 220, the datum unit 250 may form a corresponding contact 252 with the disc surface 263 and abut against the disc surface 263 in a direction substantially parallel to the gripping axis 230. As such, the datum unit not only helps to stabilize the disc 260 but also helps to level the disc 260.

Using the gripper 200 to transport an object is simple and effective. For instance, to transport the disc 260, the gripper 200 may be moved to where the disc 260 is located (e.g., in the drawer 300 or an analytic module). If necessary, the gripper 200 may be oriented so that the gripping axis 230 is substantially perpendicular to the disc surface 263 and/or substantially aligned with the aperture 261 of the disc 260. The pushing member 241 may be operated to abut outwardly against the first finger portion 221 of each respective finger 220 to cause the gripping part 223 of each respective finger 220 to move radially inward relative to the gripping axis 230. The staging unit 210 may then be moved to insert the gripping parts 223 of the plurality of fingers 220 into the aperture 261 of the disc 260. Afterward, the pushing member 241 may be moved to abut outwardly against the second finger portion 222 of each respective finger to cause the gripping part 223 of each respective finger 220 to move radially outward relative to the gripping axis 230 and engage with the step 262 of the disc 260. When the gripping parts 223 of the plurality of fingers 220 are engaged with the step 262 of the disc 260, the staging unit 210 may be moved to lift up the disc 260 and transport the disc 260 to any desired location. When the disc 260 is transported to a desired location, the pushing member 241 may be operated again to abut outwardly against the first finger portion 221 of each respective finger 220. This causes the gripping part 223 of each respective finger 220 to move radially inward relative to the gripping axis 230 and disengage from the step 262 of the disc 260. The staging unit 210 may then be moved to pull the gripping parts 223 of the plurality of fingers 220 out of the aperture 261 of the disc 260.

### Exemplary Drawer

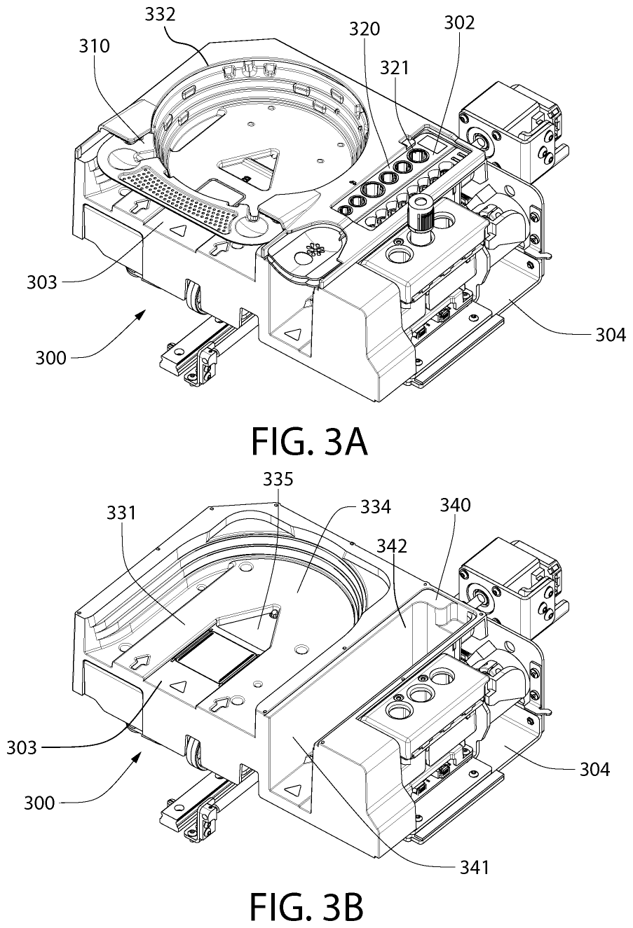

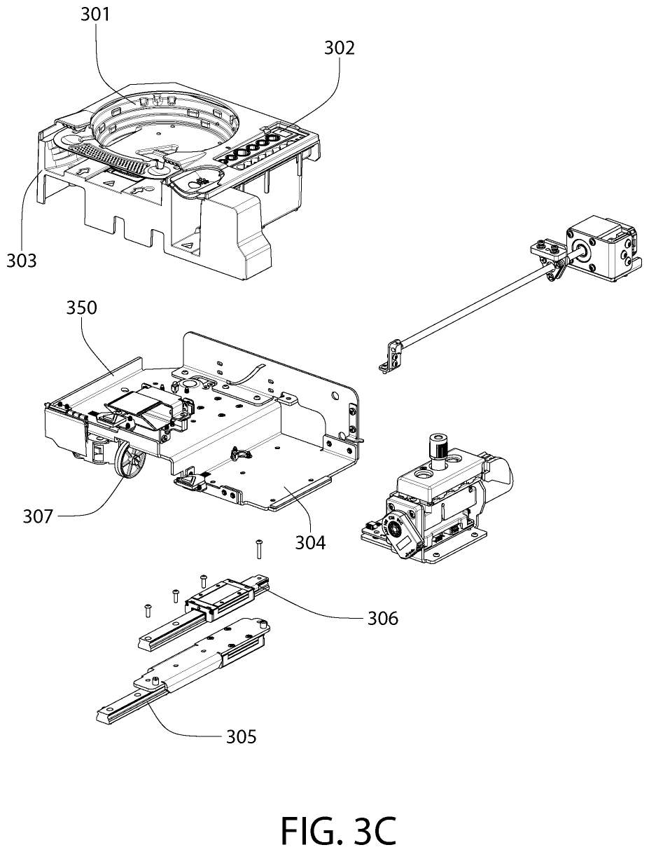

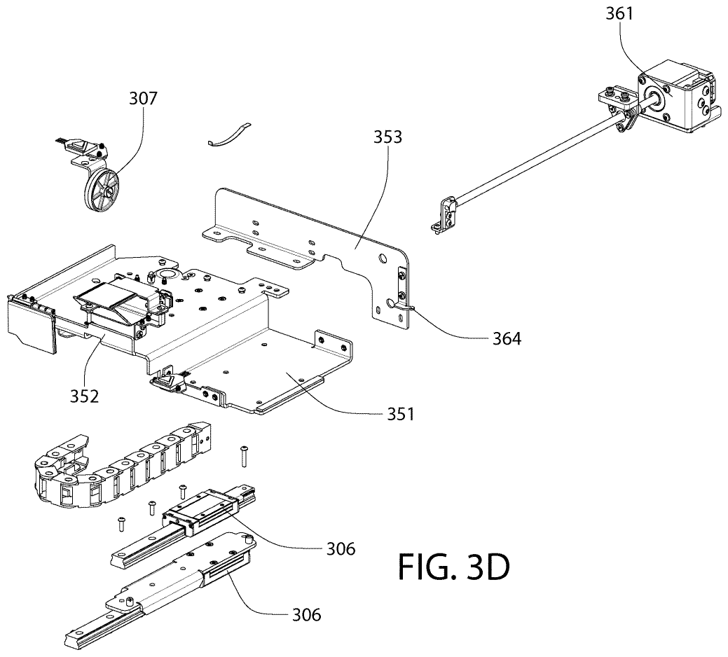

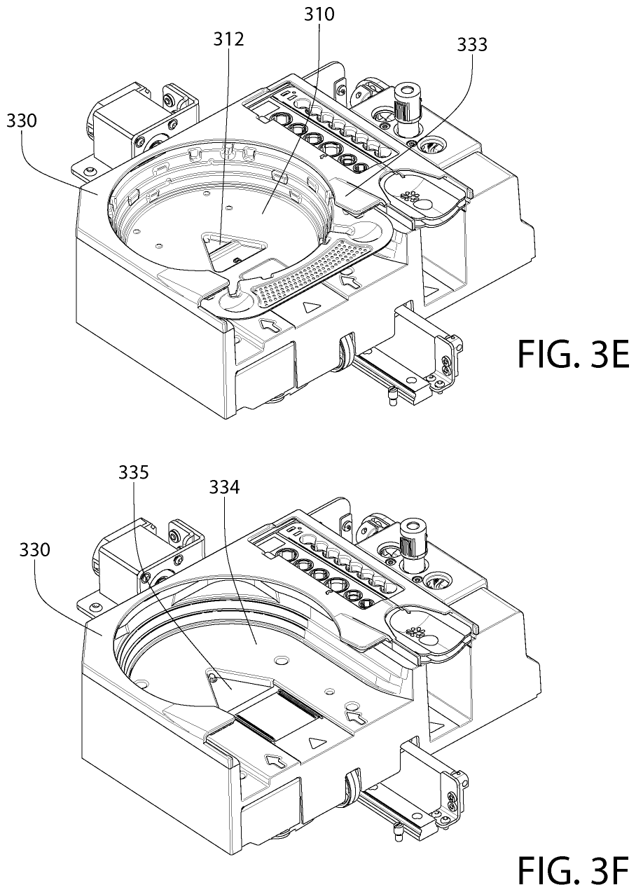

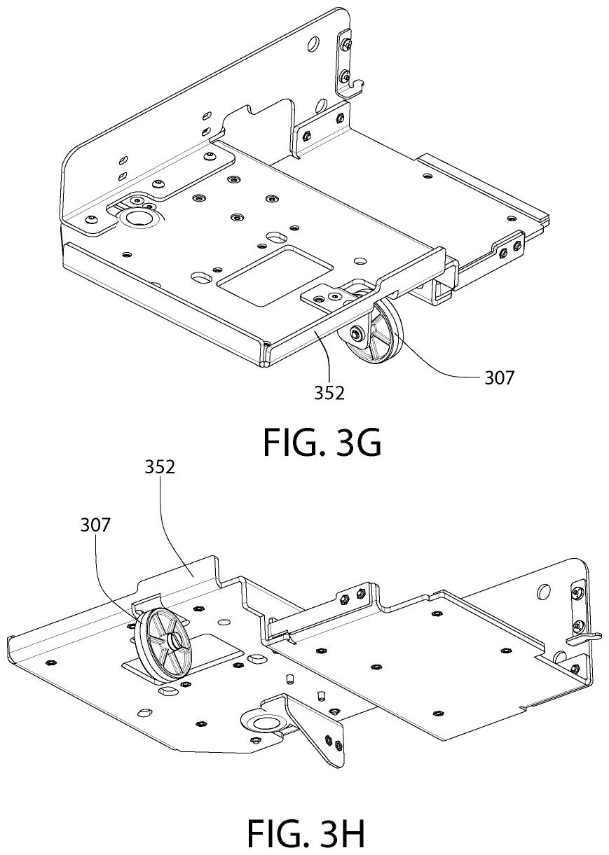

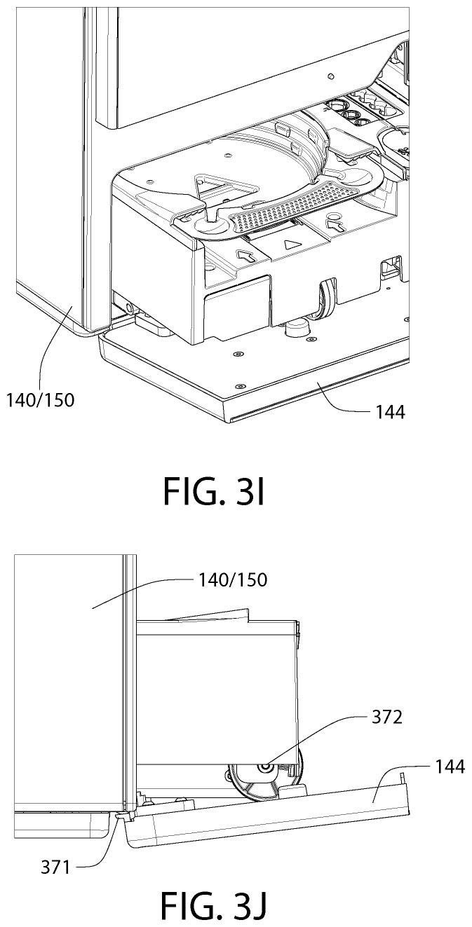

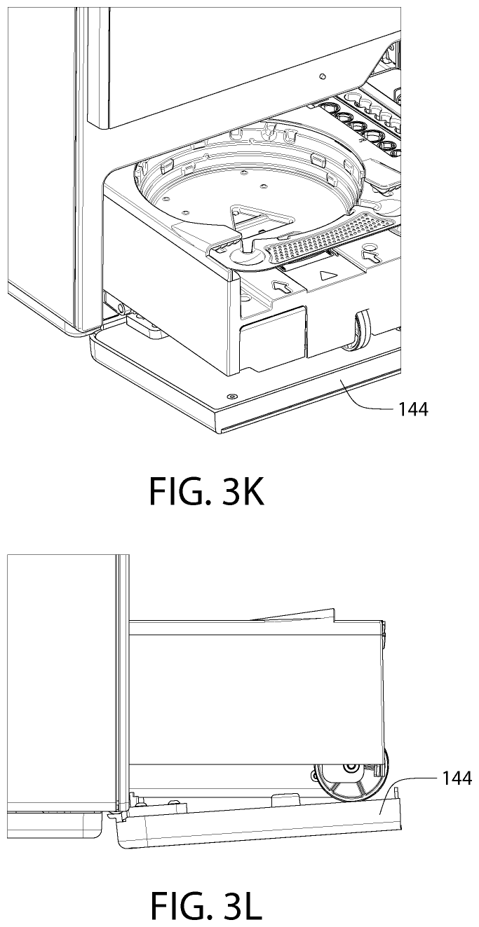

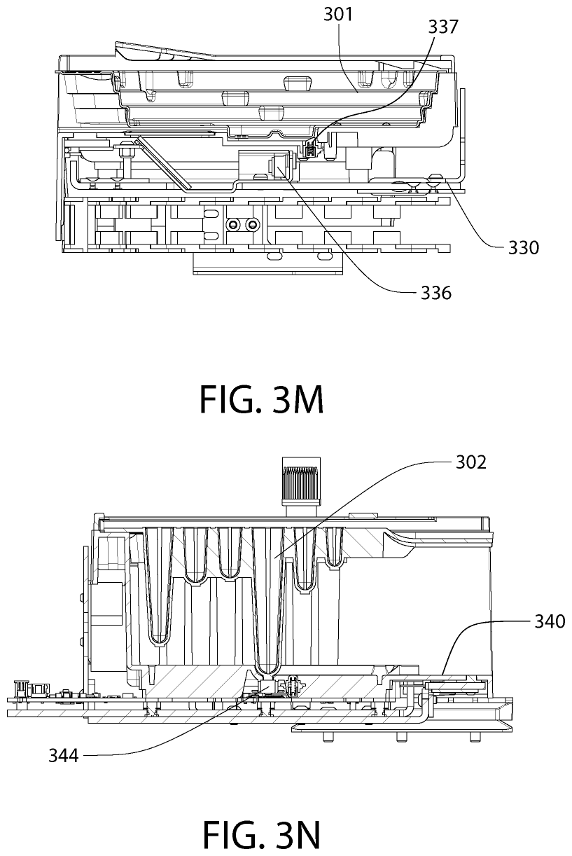

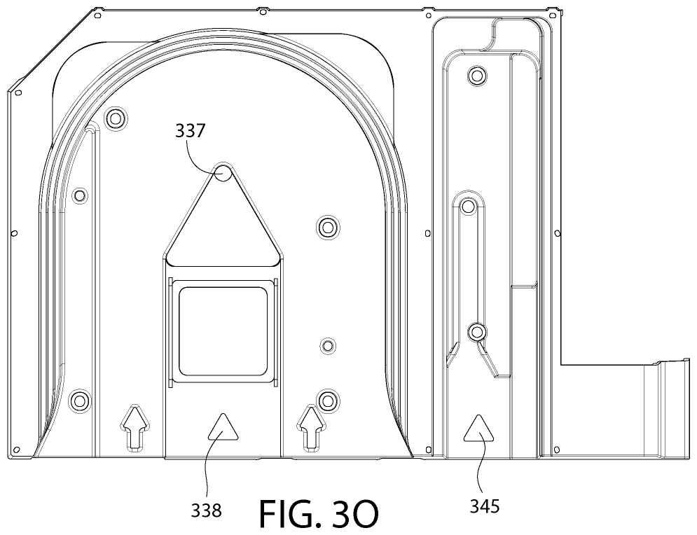

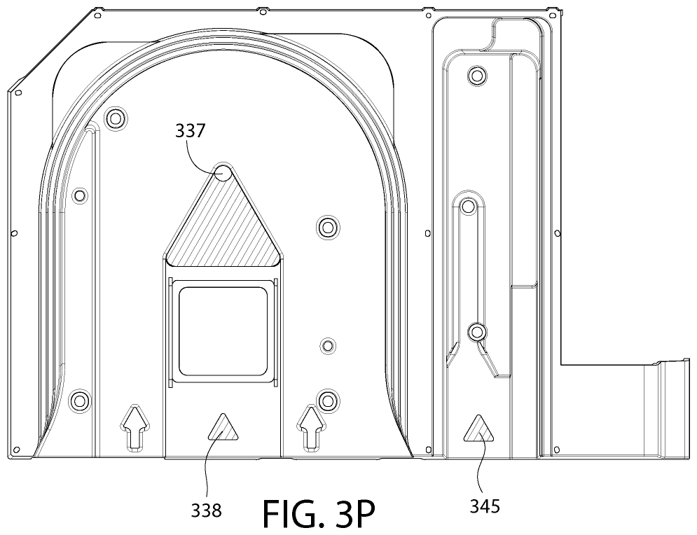

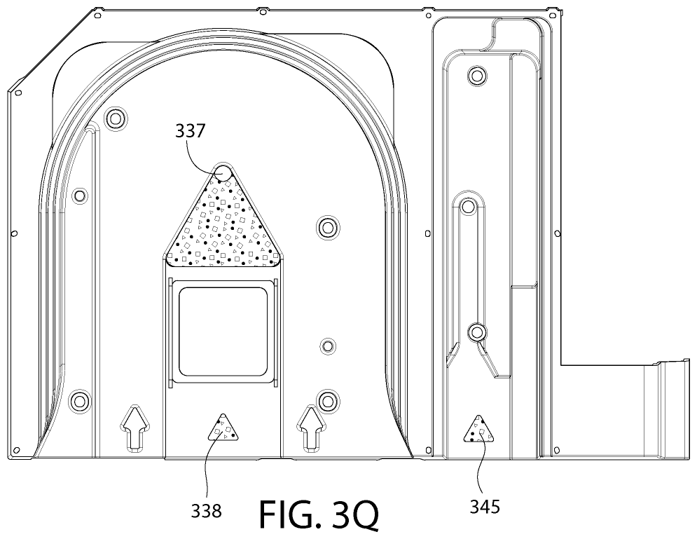

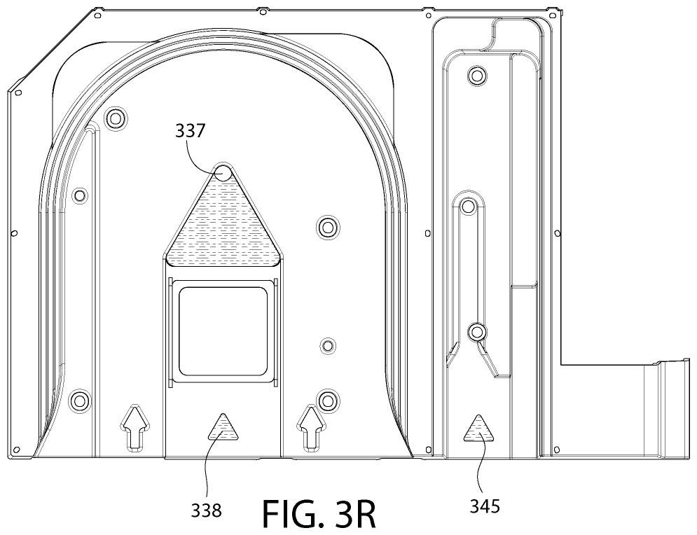

Referring to FIGS. 3A-3L, there is shown an exemplary drawer 300, along with some other components, in accordance with some embodiments of the present disclosure. The drawer 300 is configured for receiving and holding some consumables, such as a disc pack 301 and a supporting pack 302, and/or other packs or components. The disc pack 301 may include a casing 310 and one or more items 311 packed in the casing 310. The one or more items 311 may be a microfluidic disc configured for hematology analysis (e.g., for a complete blood count test), a microfluidic disc for immunoassay analysis, a microfluidic disc for clinical chemistry analysis, and a urine test strip or any combination thereof. A microfluidic disc may have an aperture, a step and a disc surface similar to those of the disc 260 so that it can be gripped and transported using the gripper 200. The supporting pack 302 may include a casing 320 and one or more items 321 packed in the casing 320. The one or more items 321 may include (e.g., one or more reagents, one or more pipette tips, or any combination thereof. Additional information regarding consumables and consumable packs can be found in U.S. Provisional Patent Application No. 63/498,228, filed Apr. 25, 2023, the content of the application is hereby incorporated by reference in its entirety.

Referring in particular to FIGS. 3A-3B and 3E-3F, the drawer 300 includes a drawer nesting unit 303 having one or more drawer nests, each configured for housing a consumable pack. For instance, in some embodiments, the drawer nesting unit 303 includes a first drawer nest 330 configured for housing the disc pack 301 and a second drawer nest 340 configured for housing the supporting pack 302. However, the present disclosure is not limited thereto. The drawer nesting unit 303 can have a single drawer nest, or have three, four, five, or more than five drawer nests. In some embodiments, the first drawer nest 330 and the second drawer nest 340 are formed as a unitary piece. In some embodiments, the entire drawer nesting unit 303 is formed of a single piece.

In some embodiments, a respective drawer nest in the one or more drawer nests includes a nest slot, a nest opening, and a nest frame. The nest slot is generally formed at a front side of the drawer nesting unit to allow insertion of a corresponding pack into the respective drawer nest. The nest opening is generally formed at an upper side of the drawer nesting unit to allow access to the one or more items contained in the corresponding pack. The nest frame is formed above the corresponding pack to prevent the casing of the corresponding pack from moving upwardly.

For instance, in some embodiments, the first drawer nest 330 includes a nest slot 331, a nest opening 332, and a nest frame 333. The nest slot 331 is generally formed at the front side of the drawer nesting unit 303 to allow insertion of the disc pack 301 into the first drawer nest 330. The nest opening 332 is generally formed at the upper side of the drawer nesting unit 303 to allow access to the one or more items 311 (e.g., disc) contained in the disc pack 301. In some embodiments, the nest opening 332 is substantially circular, or has a shape that generally matches the shape of the disc(s). The nest frame 333 is formed above the disc pack 301 to prevent the casing 310 of the disc pack 301 from moving upwardly. The nest frame 333 may be actuated, for instance, by an actuator, to restrain the disc pack 301 from moving upwardly once the disc pack 301 is inserted into the first drawer nest 330; once a test is started, when an item 311 is being retrieved from the disc pack 301; or at any other time as programmed.

Similarly, in some embodiments, the second drawer nest 340 includes a nest slot 341, a nest opening 342, and a nest frame 343. The nest slot 341 is generally formed at the front side of the drawer nesting unit 303 to allow insertion of the supporting pack 302 into the second drawer nest 340. The nest opening 342 is generally formed at the upper side of the drawer nesting unit 303 to allow access to the one or more items 321 (e.g., reagent, pipetting tip) contained in the supporting pack 302. The nest frame 343 is formed above the supporting pack 302 to prevent the casing 320 of the supporting pack 302 from moving upwardly. The nest frame 343 may be actuated, for instance, by an actuator, to restrain the supporting pack 302 from moving upwardly once the supporting pack 302 is inserted into the first drawer nest 330; once a test is started, when an item 321 is being retrieved from the supporting pack 302; or at any other time as programmed.

A drawer nest may include additional, optional or alternative components. For instance, referring in particular to FIGS. 3B, 3E and 3F, in some embodiments, the first drawer nest 330 includes a nest base 334 having a nest retaining member 335. The nest retaining member 335 is configured to engage with a retaining member 312 of the casing 310 of the disc pack 301 to restrict rotation of the casing 310 of the disc pack 301 relative to the first drawer nest 330. As such, the disc(s) contained in the casing 310 can be easily pinpointed and retrieved, for instance, by the gripper 200. The nest retaining member 335 and the retaining member 312 can be of any regular or irregular shapes, including but not limited to polygons and ovals. As a non-limiting example, the nest retaining member 335 and the retaining member 312 are shown in a substantially triangular shape.

In some embodiments, the first drawer nest 330 includes a nest camera 336 configured to retrieve information from a barcode on the casing 310 of the disc pack 301. The information may be related to the number of disc(s) in the disc pack 301, the type of the test to be performed by each disc, and/or any other information. The nest camera 336 may be disposed at any location corresponding to where the barcode on the casing 310 of the disc pack 301. For instance, if the barcode is located at the bottom of the casing 310 of the disc pack 301, the nest camera 336 may be disposed at the nest base 334. The nest camera need not directly look at the barcode. In some embodiments, mirrors may be placed between the nest camera 336 and the barcode position if size constraints exist.

Referring in particular to FIGS. 3N-3R, in some embodiments, the first drawer nest 330 includes a nest position sensor 337 and an indicator 338. The nest position sensor 337 is configured to detect a position of the disc pack 301 in the first drawer nest 330. According to the detection by the nest position sensor 337, the indicator 338 indicates whether the disc pack 301 is properly positioned in the first drawer nest 330. The detection and indication of the position of the disc pack 301 may occur once the disc pack 301 is inserted into the first drawer nest 330 and before the test is started. The indicator 338 may indicate whether the disc pack 301 is properly positioned in the first drawer nest 330 visually, aurally, or both visually and aurally. For instance, the indicator 338 may indicate whether the disc pack 301 is properly positioned in the first drawer nest 330 by color and/or blink using one or more lights (e.g., light emitting diodes). In some embodiments, the indicator 338 may indicate any incorrect position (e.g., the disc pack 301 has not been fully inserted) by a first color (e.g., yellow), the wrong disc pack 301 (e.g., expired disc pack) by a second color (e.g., red) and/or the correct position (e.g., the disc pack 301 has been correctly placed) by a third color (e.g., white). The indicator 338 may be disposed at a front or upper side of the first drawer nest 330, or any other suitable locations visible to users. As a non-limiting example, one indicator 338 is shown at the front of the first drawer nest 330 and one indicator 338 is shown below the disc pack. In some embodiments, the nest camera 336 is used as the nest position sensor.

Similarly, in some embodiments, the second drawer nest 340 includes a nest position sensor 344 and an indicator 345. The nest position sensor 344 is configured to detect a position of the supporting pack 302 in the second drawer nest 340. According to the detection by the nest position sensor 344, the indicator 345 indicates whether the supporting pack 302 is properly positioned in the second drawer nest 340. The detection and indication of the position of the supporting pack 302 may occur once the supporting pack 302 is inserted into the second drawer nest 340 and before the test is started. The indicator 345 may indicate whether the supporting pack 302 is properly positioned in the second drawer nest 340 visually, aurally, or both visually and aurally. For instance, the indicator 345 may indicate whether the supporting pack 302 is properly positioned in the second drawer nest 340 by color and/or blink using one or more lights (e.g., light emitting diodes). In some embodiments, the indicator 345 may indicate any incorrect position (e.g., the supporting pack 302 has not been fully inserted) by a first color (e.g., yellow), the wrong supporting pack 302 (e.g., expired supporting pack) by a second color (e.g., red) and/or the correct position (e.g., the supporting pack 302 has been correctly placed) by a third color (e.g., white). The indicator 345 may be disposed at a front or upper side of the second drawer nest 340, or any other suitable locations visible to users. As a non-limiting example, the indicator 345 is shown at the front of the second drawer nest 340.

Referring in particular to FIGS. 3A-3D, in some embodiments, the drawer 300 includes a drawer structure unit 304 having a drawer chassis 350 configured for mounting the drawer nesting unit 303. In some embodiments, the drawer chassis 350 includes a body 351, a first member 352 at a front side of the body 351, and a second member 353 at a back side of the body 351. The first member 352 and second member 353 may be fixed or formed with the body 351. Additionally, or optionally, the drawer chassis 350 may be configured for mounting one or more other components, such as a homing member (e.g., homing flag and sensor) for detecting the position of the drawer and/or other components of the system, and a cam member 363 and a cam member 364 for engaging with other components of the system.

In some embodiments, the drawer 300 includes one or more drawer rails 305 and one or more drawer rail carriages 306 to allow the drawer chassis 350 to slide along the one or more drawer rails 305. The one or more drawer rails 305 and one or more rawer rail carriages 306 may be disposed in the housing 140 adjacent to the door 144 so that at least a portion of the drawer 300 can be moved out of the housing 140 to allow loading and unloading of the consumables. Alternatively, in some embodiments, the drawer 300 does not include a rail or rail carriage; instead, the drawer structure unit 304 is configured for coupling with a rail in the housing 140 adjacent to the door 144.

Referring in particular to FIGS. 3G-3L, in some embodiments, a roller 307 is connected to the first member 352 of the drawer chassis 350. The door 144 is connected to the housing 140 or the chassis and gantry unit 150 by one or more spring hinges. The door 144 is rotatable relative to the housing 140 around a door hinge axis 371. The drawer chassis 350 is slidable in a direction substantially perpendicular to the door hinge axis 371. Because the drawer roller 307 is connected to the first member 352 of the drawer chassis 350, the drawer roller 307 is able to move along with the drawer chassis 350 and rotatable around a roller rotational axis 372 that is substantially parallel to the door hinge axis 371. As the drawer chassis 350 slides toward the door, the drawer roller 307 rolls on an inner surface of the door and thus pushes the door to open. As the drawer chassis 350 slides away from the door, the one or more spring hinges pulls and closes the door. The drawer chassis 350 may slide toward the door in response to a command from a user (e.g., by scanning their badge to start a test) or when the test is completed. The drawer chassis 350 may slide away from the door in response to a command from a user or upon detection of proper positioning of the consumables. With the roller 307, opening and closing the door is simple, smooth, and reliable.

### Exemplary Tube Holder and Tube Rocker

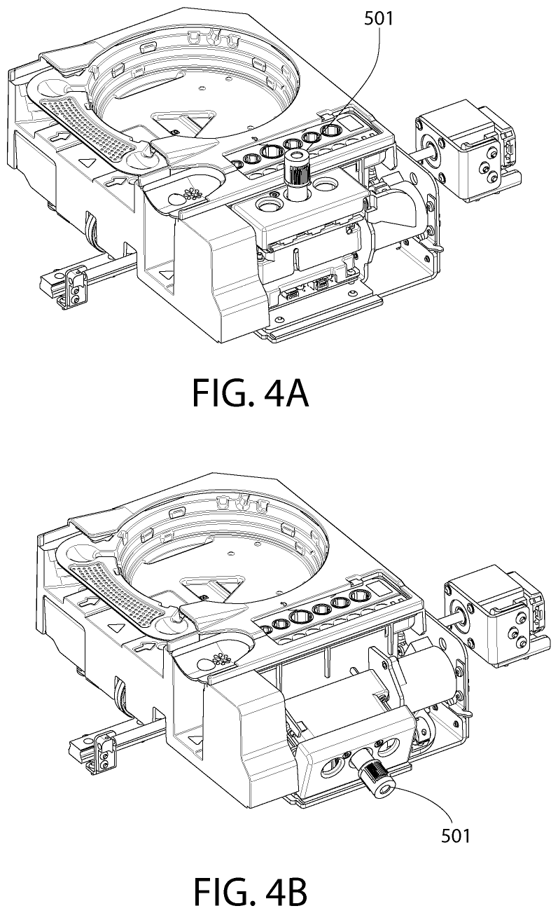

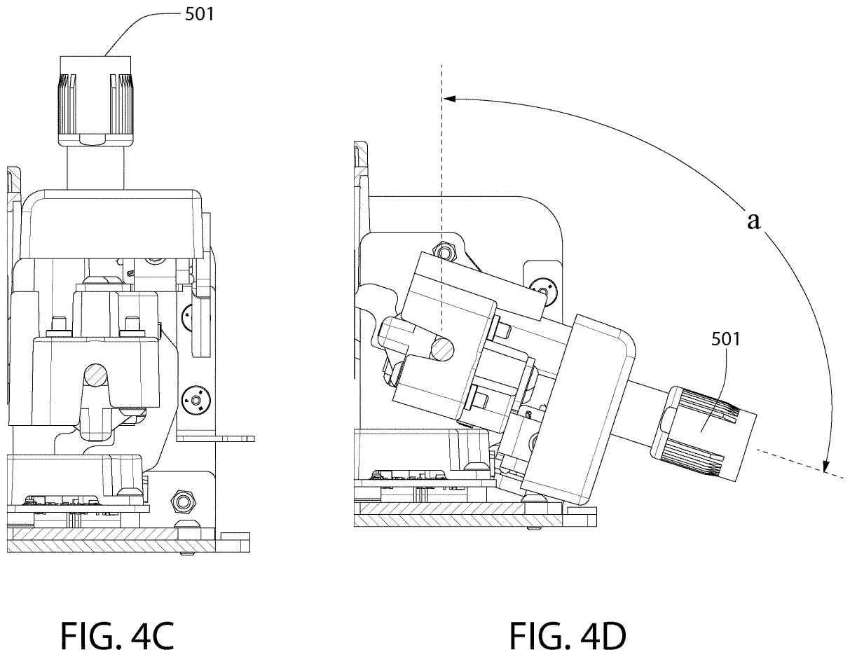

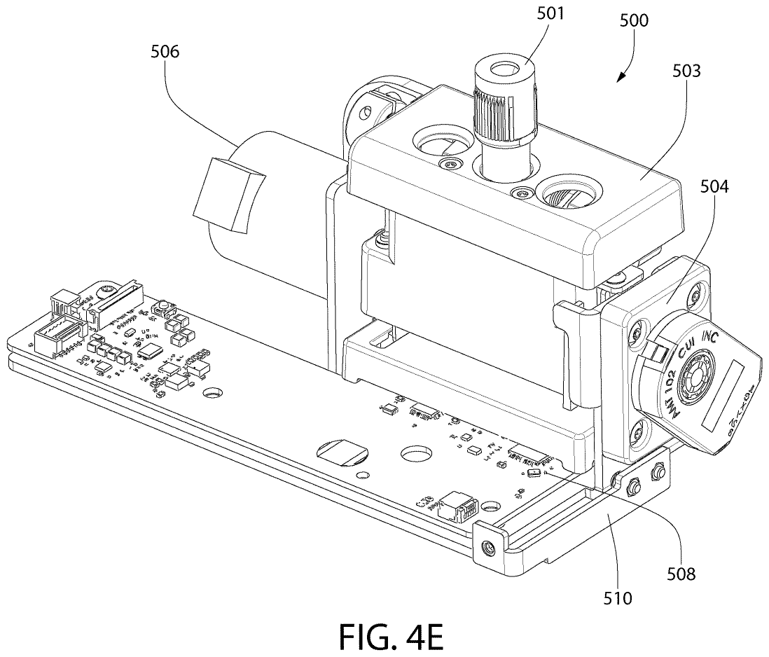

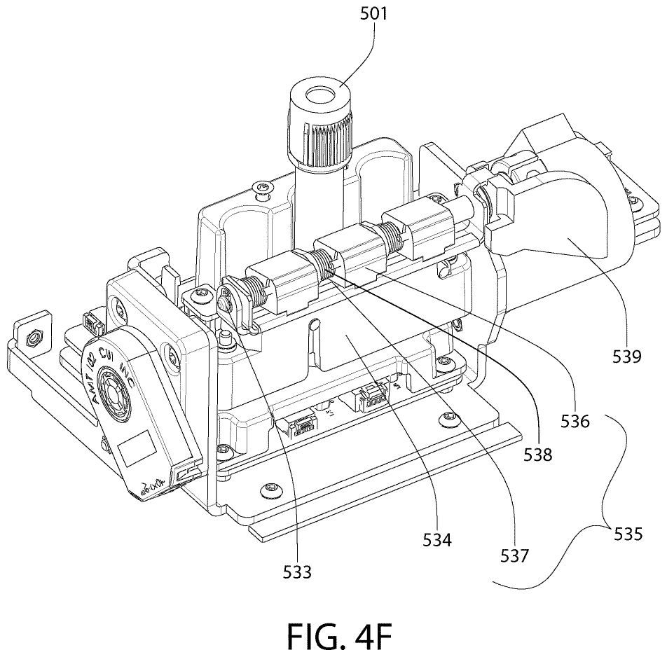

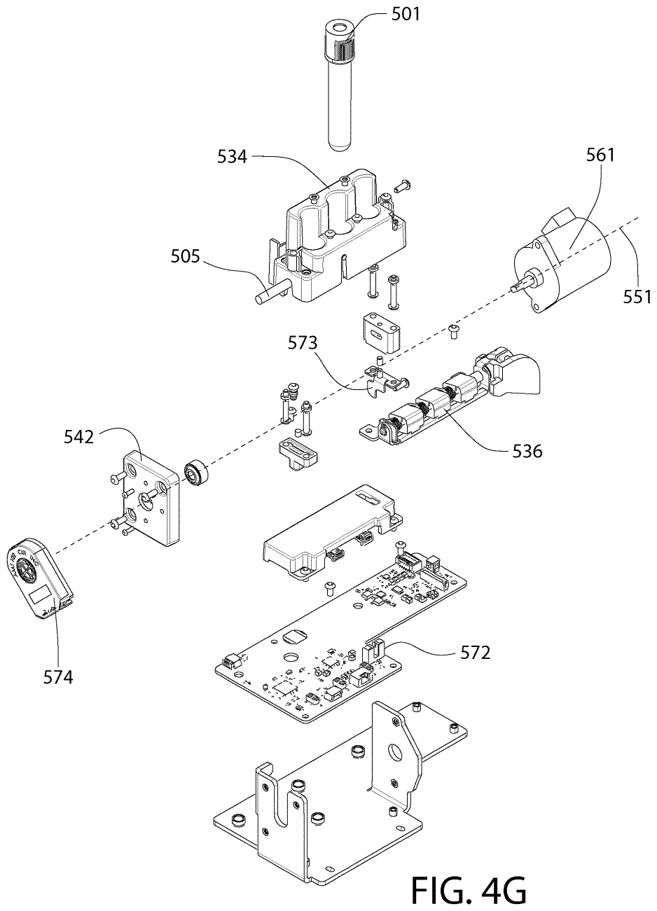

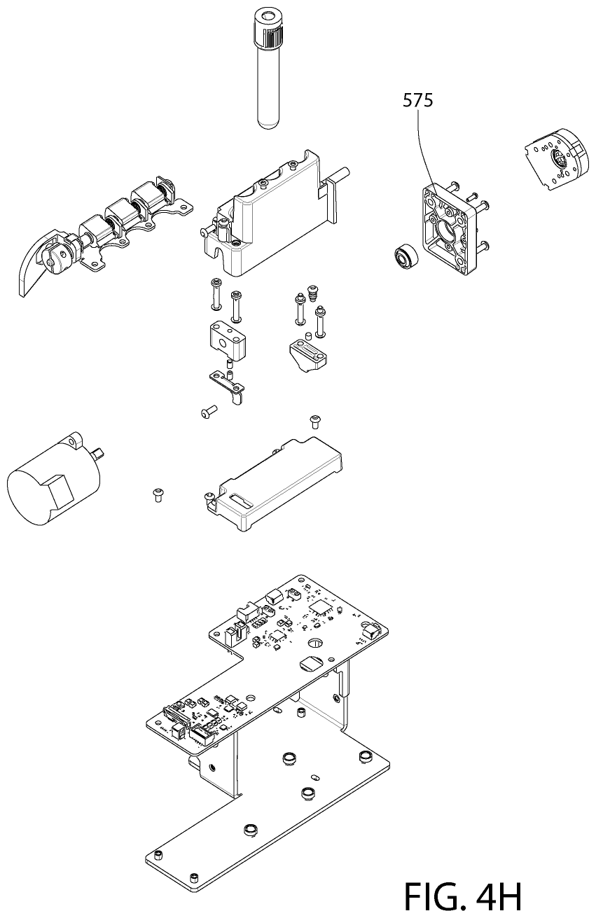

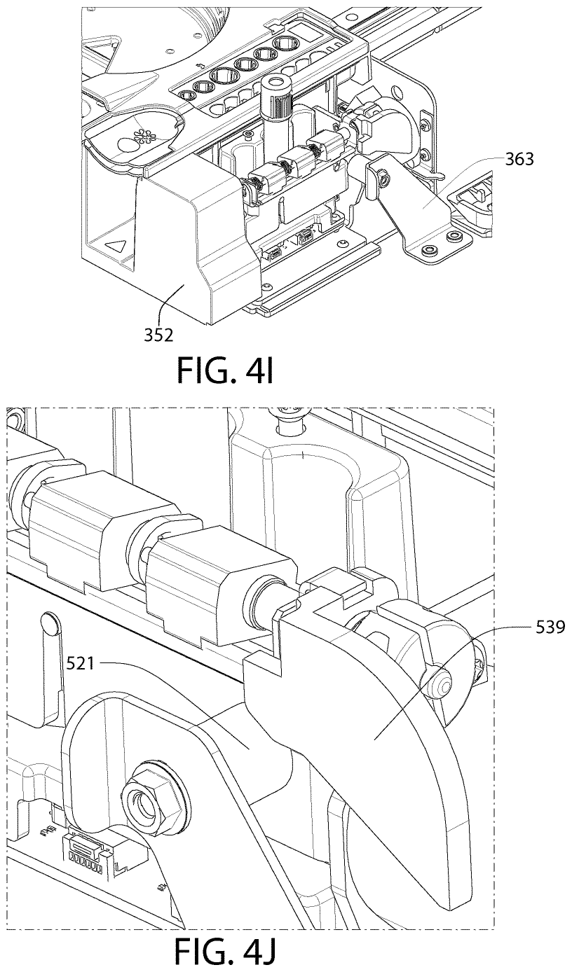

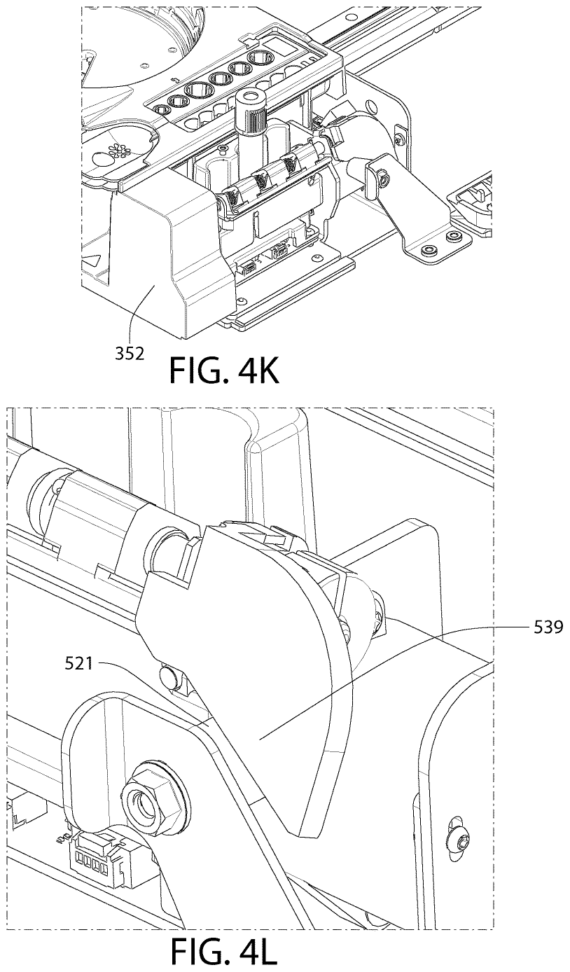

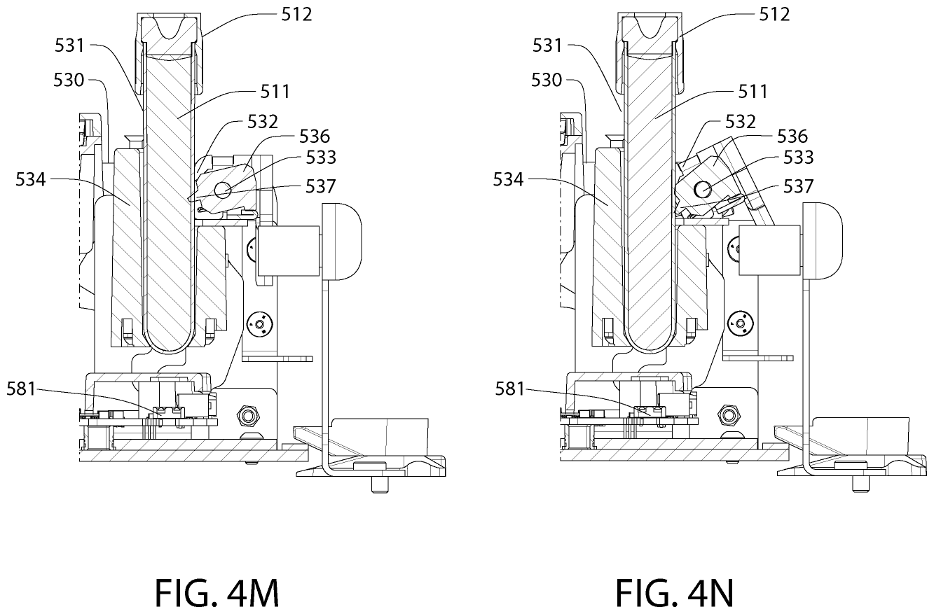

Referring to FIGS. 4A-4N, there is shown an exemplary tube rocker 500, along with some other components, in accordance with some embodiments of the present disclosure. The tube rocker 500 is configured for rocking (e.g., rotation or swing) any sample tube 501 to mix the sample contained in the sample tube 501. The tube rocker 500 generally includes a tube holder 503, a rocker mounting unit 504, a rocker shaft 505 and a rocker driving unit 506. The tube holder 503 is configured for holding one or more sample tubes 501. The rocker shaft 505 has a rotational axis 551 and rotatably mounted on the rocker mounting unit 504. In some embodiments, the rocker shaft 505 is configured for fixedly connecting the tube holder 503. The rocker driving unit 506 is connected to the rocker shaft 505 and configured to rotate the rocker shaft 505 clockwise and counter-clockwise around its rotation axis 551 between a positive angle (e.g., α) and a negative angle (e.g., −α). Consequently, this rocks the tube holder 503 and any sample tube 501 held by the tube holder 503 between the positive angle (e.g., α) and the negative angle (e.g., −α) as illustrated in FIGS. 4A-4D. In some embodiments, the maximal allowable angle is at least 50°, at least 60°, at least 70°, at least 80°, at least 90°, at least 100°, at least 110°, or at least 120°. In some embodiments, the maximal allowable angle is at most 160°, at most 150°, at most 140°, at most 130°, at most 120°, at most 110°, or at most 100°.

The rocking may be performed, for instance, at a controlled speed, a controlled acceleration, a controlled deceleration, a controlled frequency, a controlled number of times, or any combination thereof. The control of the rocking may be determined based on the type of the sample contained in the sample tube(s) and/or the test to be performed. In some embodiments, the rocking is performed to drive any bubbles down to the bottom of the tube without causing any adverse effect to the sample (e.g., without causing more shear stress on the cells of the sample).

Referring in particular to FIGS. 4E-4N, in some embodiments, the tube holder 503 includes a rack 530 having one or more rack slots 531, each configured for housing a tube body 511 of a sample tube 501. By way of illustration, three slots and three tubes are shown. However, the present disclosure is not limited thereto. The rack 530 can have any suitable number of slots, including but not limited to one, two, three, four, five or more than five slots. Moreover, each slot can house, but does not necessarily have to house, any sample tube 501. For instance, if a test requires only a single sample tube, two of the three slots will not house any sample tube. If a test requires two sample tubes, one of the three slots will not house any sample tube. Further, multiple sample tubes can be, but do not necessarily have to be, placed into the tube holder 503 in any specific order. For instance, in some embodiments, the tube rocker 500 or the system 100 may include a detection unit configured for distinguishing a sample tube from another sample tube. A non-limiting example is that the caps 512 of sample tubes 501 may be in different colors indicating different types of samples (e.g., blood or urine) or anticoagulants (e.g., anticoagulant for hematology or anticoagulant for immunoassay or clinical chemistry) in the sample tubes 501. The detection unit may detect the colors and determine which slot houses what type of samples. This reduces user errors.

The cap 512 of each sample tube 501 may be disposed outside of the corresponding rack slot of the rack 530, and coupled with the tube body 511 by an interference fit or the like. The rack 530 is open at least partially at a first side 532 of the rack 530. This allows access to at least a portion of the tube body 511 of the sample tube 501 housed in any of the one or more rack slots 531.

In some embodiments, the tube holder 503 also includes a holder shaft 533 disposed adjacent to the first side 532 of the rack 530. For instance, in some embodiments, the rack 530 and the holder shaft 533 are mounted on a holder body 534 with the first side 532 of the rack 530 facing the holder shaft 533.

In some embodiments, the tube holder 503 further includes one or more retaining units 535. Each of the one or more retaining units 535 is associated with a corresponding rack slot in the one or more rack slots 531 of the rack 530. For instance, as a non-limiting example, three retaining units 535 are illustrated, each corresponding to one of the three rack slots 531. Each respective retaining unit 535 is configured for retaining the tube body 511 of the sample tube 501 in the corresponding rack slot 531 of the rack 530. In some embodiments, a respective retaining unit 535 includes a tube retaining member 536 and a holder elastic member 538. The tube retaining member 536 is rotatably disposed at the holder shaft 533 and includes an edge at a side facing the first side 532 of the rack 530. In some embodiments, the tube retaining member 536 of each retaining unit 535 is independently rotatable around the holder shaft 533. The holder elastic member 538 biases the tube retaining member 536 to push the edge against the tube body 511 of the sample tube 501 housed in the corresponding rack slot 531, thereby retaining the tube body 511 of the sample tube 501 in the corresponding rack slot 531 of the rack 530.

The edge 537 of the tube retaining member 536 can have any suitable shapes and sizes. For instance, the edge 537 of the tube retaining member 536 may be a wedge, a knife edge, a knurled surface or the like that can provide frictional contact with the tube body 511. The holder elastic member 538 can be any suitable type that can provide necessary force to the tube retaining member 536. For instance, in some embodiments, the holder elastic member 538 is a coil spring having a first end connected to the rack 530 or a stationary component and a second end connected to (e.g., abutting against) the tube retaining member 536.

In some embodiments, the one or more rack slots 531 comprise a plurality of rack slots, and the one or more retaining units 535 comprise a plurality of retaining units. In some embodiments, the plurality of rack slots comprises at least three rack slots, and the plurality of retaining units comprises at least three retaining units. In some embodiments, the tube holder 503 is configured for housing a first sample tube containing a first type of blood, a second sample tube containing a second type of blood, a third sample tube containing urine, or any combination thereof. The first type of blood may be ethylenediaminetetraacetic acid (EDTA) anticoagulated blood. The second type of blood may be lithium heparin anticoagulated blood. In some embodiments, the tube holder 503 may be configured to hold one or more sample tubes of smaller diameters (e.g., microcontainers), each via a tube adapter. For instance, a small sample tube may be inserted into a tube adapter and then the adapter along with the sample tube may be inserted into the tube holder 503 and/or other devices/mechanisms and used in a way similar to the sample tubes 501.

Referring in particular to FIGS. 4I-4N, the tube holder 503 may include one or more cam surfaces configured for engaging/disengaging the edge 537 of the tube retaining member 536 of each retaining unit 535 with/from any sample tube 501 that has been inserted in any rack slot 531 of the rack 530. For instance, in some embodiments, the tube holder 503 includes a holder cam surface 539 connected to the holder shaft 533 and configured for abutting against an external component 521. This rotates the tube holder 503 and thus disengages the edge 537 of the tube retaining member 536 of each retaining unit 535 from the sample tube 501 that has been inserted in the corresponding rack slot 531. For instance, as illustrated in FIGS. 4I, 4J and 4M, when the holder cam surface 539 does not abut against the external component 521, the tube retaining member 536 of each retaining unit 535 is biased by the holder elastic member 538 so that the edge 537 of the tube retaining member 536 engages with the sample tube 501 that has been inserted in the corresponding rack slot 531. The engagement of the edge 537 of the tube retaining member 536 of each retaining unit 535 with the sample tube 501 provides necessary friction force to retain the sample tube 501 in the corresponding rack slot 531. This prevents the sample tube 501 from slipping out of the rack 530 during the rocking or other process. As illustrated in FIGS. 4K, 4L and 4N, when the holder cam surface 539 abuts against the external component 521, it rotates the tube holder 503 so that the edge 537 of the tube retaining member 536 of each retaining unit 535 disengages from the sample tube 501 that has been inserted in the corresponding rack slot 531. This provides clearance that allows easy insertion of the sample tube(s) into the rack 530 and easy removal of the sample tube(s) from the rack 530. The external component 521 may be mounted on the drawer chassis 350 at or adjacent to the first member 352 or any other suitable place.

Referring in particular to FIGS. 4E-4H, in some embodiments, the rocker mounting unit 504 comprises a mounting member 542 spaced apart in a direction substantially parallel to the rotation axis 551. The rocker shaft 505 is rotatably connected to the mounting member 542. As a non-limiting example, it is illustrated that the rocker shaft 505 is rotatably connected to the mounting member 542. The tube holder 503 is disposed on the mounting member 542, and connected to the rocker shaft 505, for instance, through the holder body 534.

The rocker driving unit 506 includes a rocker motor 561 connected to the rocker shaft 505. The rocker motor 561 may be any suitable type of motor, including but not limited to a stepper motor. In some embodiments, the rocker motor 561 is a stepper motor and the rocker shaft 505 is the rotor shaft of the stepper motor. In some embodiments, the rocker driving unit 506 also includes a coupler configured for selectively connecting the rocker shaft 505 to the tube holder 503. The rocker motor 561 is operable to rotate the rocker shaft 505 clockwise and counter-clockwise around its rotation axis 551 between a positive angle (e.g., α) and a negative angle (e.g., −α). Consequently, if the tube holder 503 is connected to the rocker shaft 505, this rocks the tube holder 503 and any sample tube 501 held by the tube holder 503 between the positive angle (e.g., α) and the negative angle (e.g., −α) as illustrated in FIGS. 4A-4D.

The rocker 500 or the rocker driving unit 506 may include one or more additional or optional components to facilitate the rocking of the tube holder 503 and any sample tube 501 held by the tube holder 503 in a controlled and/or safe manner. For instance, additionally or optionally, the rocker driving unit 506 may include a rocker detent 575, a rocker detent guide 571, a limit switch 572, a limit switch flag 573, or similar components, or any combination thereof to set the limits for the swinging angles, to prevent the swinging past the limits, to switch the rotation direction when reaching the set limits, or any combination thereof. The rocker detent 575, the rocker detent guide 571, the limit switch 572, and/or the limit switch flag 573 may be disposed at or adjacent to the mounting member 542 of the mounting unit 504. Additionally, or optionally, the rocker driving unit 506 may include an encoder 574 to provide feedback signals by tracking the speed and/or position of the rocker shaft 505. The encoder 574 may be mounted on the rocker motor 561 and configured to track the speed and/or position of the rocker shaft 505 and provide feedback signals.

Referring in particular to FIG. 4E, in some embodiments, the rocker 500 includes a rocker sensing unit 508 configured to detect a position of the one or more sample tubes contained in the tube holder 503, to determine if any of the one or more sample tubes 501 is positioned improperly, or both. The rocker sensing unit 508 may include one or more sensors 581 disposed below the rocker shaft 505. In some embodiments, corresponding to each of the one or more rack slots 531, the rocker sensing unit 508 includes at least one sensor 581. For instance, in the illustrated embodiment, the rocker sensing unit 508 includes three sensors 581, one corresponding to each of the three rack slots 531 and configured for detecting the position of the sample tube 501 that has been inserted into the corresponding rack slot 531.

### Exemplary Capping/Decapping Device

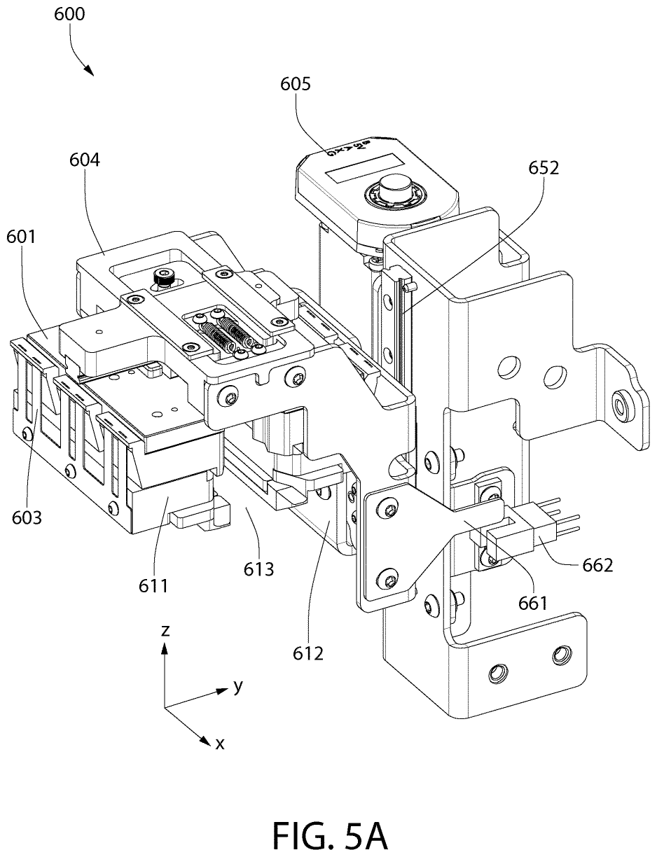

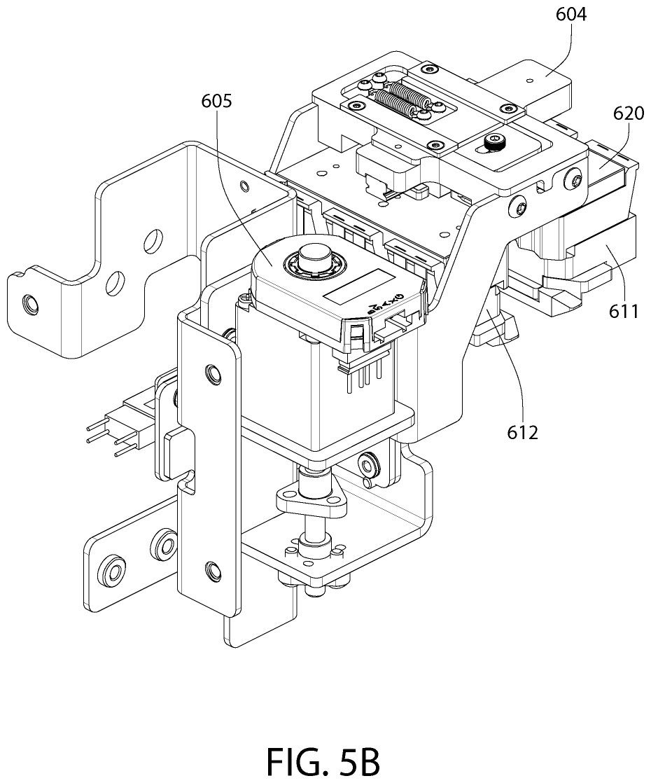

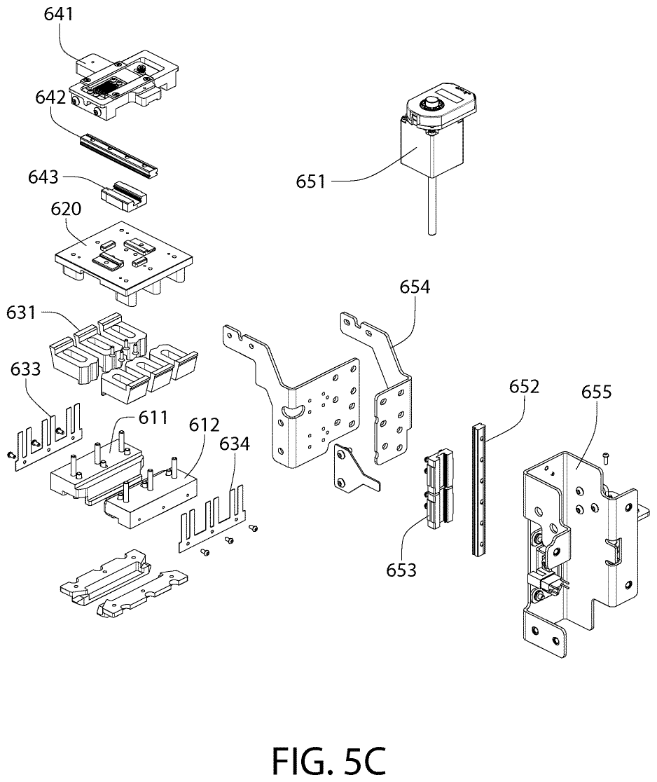

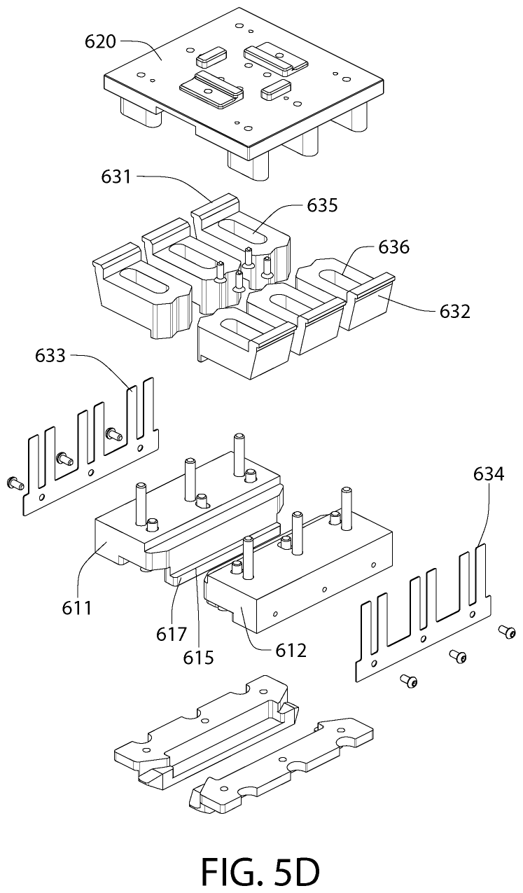

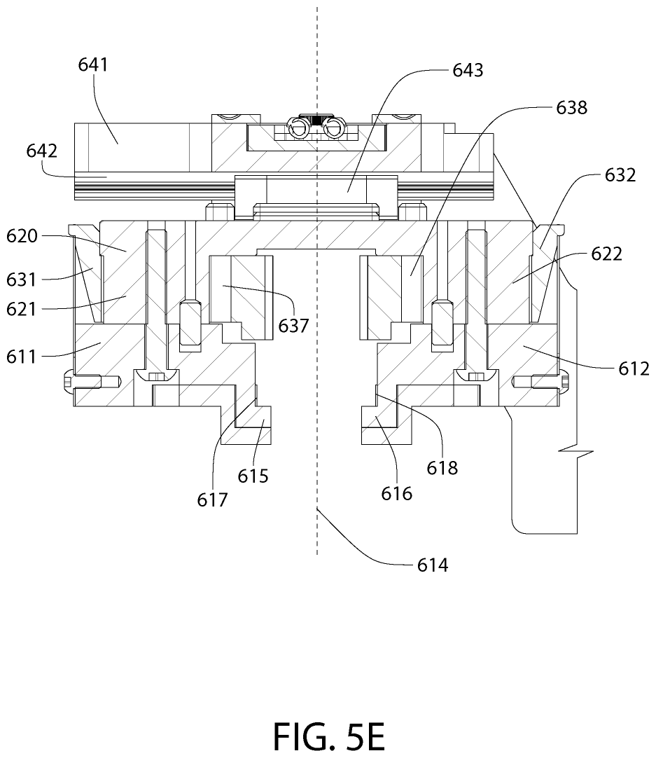

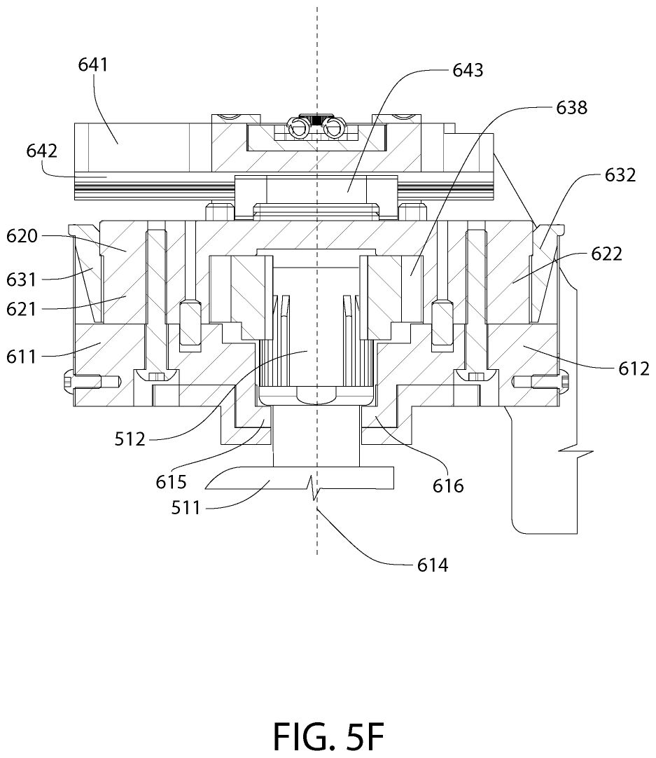

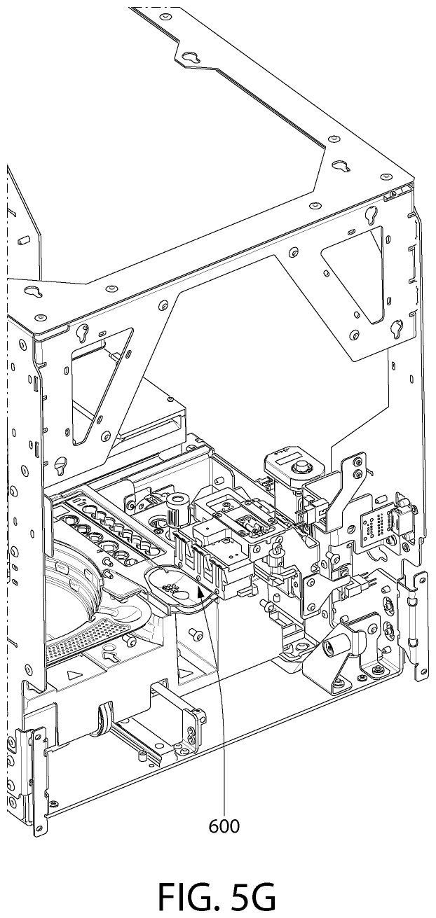

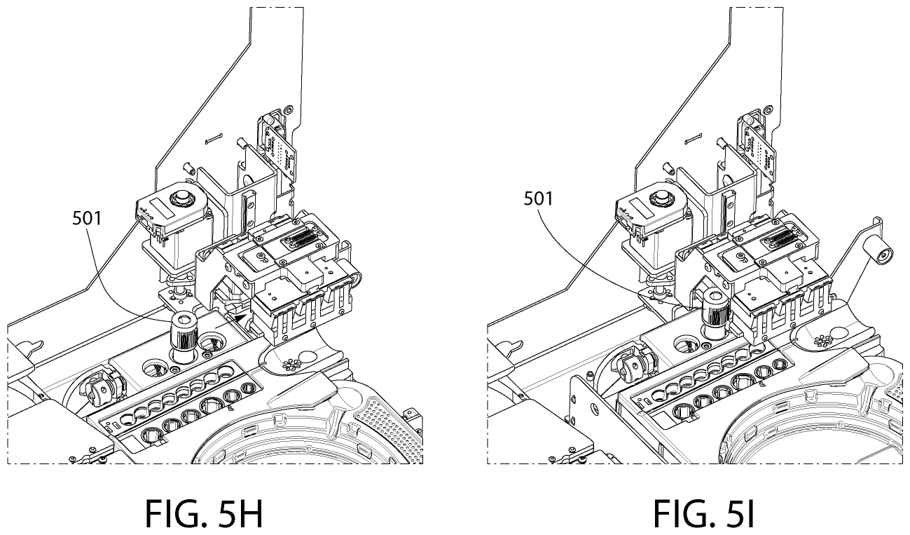

Referring to FIGS. 5A-5I, there is shown an exemplary capping/decapping device 600 in accordance with some exemplary embodiments of the present disclosure. The capping/decapping device 600 is configured for decapping one or more tubes, such as the sample tube 501 after the sample has been properly mixed by the rocker 500 or at any other time when desired. In some embodiments, the capping/decapping device 600 is also configured for recapping one or more tubes, for instance, after the use of the sample, the completion of the test, or at any other time when desired. In some embodiments, the capping/decapping device 600 is capable of simultaneously decapping a plurality of sample tubes and/or simultaneously recapping a plurality of sample tubes. However, it should be noted that the capping/decapping device 600 can, but does not necessarily have to, operate at its full capacity. For instance, in embodiments where the capping/decapping device 600 is capable of simultaneously decapping three sample tubes, the capping/decapping device 600 can be operated to decap a single sample tube, two sample tubes at a time, or all three sample tubes simultaneously.

The capping/decapping device 600 includes a cap-holding unit 601 for receiving one or more caps 512. In some embodiments, the cap-holding unit 601 includes a first segment 611 and a second segment 612. The first and second segments are spaced apart from each other in a first decapper direction (e.g., the y-direction in FIG. 5A) and collectively form a channel 613 in between. The channel 613 allows one or more caps of one or more sample tubes contained in a holder, such as the one or more caps 512 of the one or more sample tubes 501 contained in the tube holder 503, to slide into the cap-holding unit 601. The sliding of the cap(s) into the cap-holding unit 601 is generally in a second decapper direction (e.g., the x-direction in FIG. 5A) that is substantially perpendicular to the first decapper direction. The sliding of the cap(s) into the cap-holding unit 601 may be performed, for instance, using the sliding mechanism 400 disclosed herein as illustrated in FIGS. 5H and 5I. The sliding of the cap(s) into the cap-holding unit 601 may also be performed so that only a subset of caps (e.g., one or two caps out of the three caps in FIGS. 5H and 5I) is slid into the channel 613 when desired (e.g., in consideration of a specific application and/or process) or until the cap of each sample tube contained in the tube holder is slid into the channel 613.

Referring in particular to FIGS. 5A and 5D-5F, in some embodiments, the first segment 611 is formed or coupled with a first shoulder 615 at a side of the first segment 611 facing the channel 613. The first shoulder 615 is configured to allow a first portion of a lower side of the one or more caps to sit on the first shoulder 615. Similarly, in some embodiments, the second segment 612 is formed or coupled with a second shoulder 616 at a side of the second segment 612 facing the channel 613 (e.g., the first and second shoulders face each other). The second shoulder 616 is configured to allow a second portion of the lower side of the one or more caps to sit on the second shoulder 616.

In some embodiments, the first shoulder 615 is formed with one or more first indents 617, each configured for the first portion of one cap to sit. In some embodiments, the first shoulder 615 is formed with a plurality of first indents 617 (e.g., 2, 3, 4, 5, or more than 5 indents) that are disposed along the second decapper direction. Similarly, in some embodiments, the second shoulder 616 is formed with one or more second indents 618, each configured for the second portion of one cap to sit. Each of the one or more indents of the second shoulder 616 is generally aligned with a corresponding indent in the one or more indents of the first shoulder 615 along the first decapper direction. In some embodiments, the second shoulder 616 is formed with a plurality of second indents 618 (e.g., 2, 3, 4, 5, or more than 5 indents) that are disposed along the second decapper direction.

In some embodiments, the first and second shoulders allow for any cap 512, once separated from the tube body (e.g., decapped), to sit on the first and second shoulders by gravity. As a cap sits on the first and second shoulders, the first and second shoulders abut against the lower side of the one or more caps in a third decapper direction (e.g., the z-direction in FIG. 5A or the vertical direction in FIG. 5F) that is substantially perpendicular to both of the first and second decapper directions. In some embodiments, the first and second shoulders hold any cap once it is decapped from a sample tube. In some embodiments, while decapping (e.g., when a cap is pulled upward), the first and second shoulders function as a stopper to prevent the tube body 511 from moving upward.

The capping/decapping unit includes one or more cap-centering units 603, each configured for centering a cap 512 in the channel 613 at a position in accordance with a central plane 614 of the channel 613. In some embodiments, each of the one or more cap-centering units 603 is aligned with a first indent 617 on the first shoulder 615 and a second indent 618 on the second shoulder 616 along the first decapper direction. The one or more cap-centering units 603 help to properly position any cap 512 once it is slid into the channel 613 and thus ensure effective and reliable capping/decapping of the sample tube(s).

The capping/decapping unit can include any suitable number of cap-centering units 603, including but not limited to one, two, three, four, five, or more than five cap-centering units. In embodiments where there are multiple cap-centering units, the cap-centering units 603 are disposed along the second decapper direction. The cap-centering units 603 can be, but do not necessarily have to be identical to each other. As a non-limiting example, three substantially identical cap-centering units 603 are illustrated.

In some embodiments, the cap-centering unit 603 includes a first jaw 631 and a second jaw 632. The first jaw 631 is disposed at the first segment 611 of the cap-holding unit 601 above the first shoulder 615 and slidable relative to the first segment 611 in the first decapper direction (e.g., back and forth from left to right in FIG. 5E). The second jaw 632 is disposed at the second segment 612 of the cap-holding unit 601 above the second shoulder 616 and slidable relative to the second segment 612 in the first decapper direction.

In some embodiments, the cap-centering unit 603 also includes a first centering elastic member 633, and a second centering elastic member 634. The first centering elastic member 633 is configured to push the first jaw 631 along the first decapper direction towards the central plane 614 of the channel 613, and the second centering elastic member 634 is configured to push the second jaw 632 along the first decapper direction towards the central plane 614 of the channel 613. By moving towards the central plane 614 of the channel 613 from the opposite sides of the central plane 614, the first and second jaws help to align the cap 512 with the central plane 614 of the channel 613.

The first centering elastic member 633 may be disposed at or fixedly connected to the first segment 611. For instance, in some embodiments, the first centering elastic member 633 is a leaf spring having an end portion fixedly connected to the first segment 611 and another end portion abutting against the first jaw 631 towards the central plane 614 of the channel 613. Similarly, the second centering elastic member 634 may be disposed at or fixedly connected to the second segment 612. For instance, in some embodiments, the second centering elastic member 634 is a leaf spring having an end portion fixedly connected to the second segment 612 and another end portion abutting against the second jaw 632 towards the central plane 614 of the channel 613.

The cap-holding unit 601 or the cap-centering unit 603 may include additional or optional components. For instance, in some embodiments, the cap-holding unit 601 includes a third segment 620 formed or coupled with the first and second segments. The third segment 620 may be disposed on top of the first and second segments with the first and second jaws in between. This ensures the smooth movement of the first and second jaws (e.g., preventing the first and second jaws from moving the third decapper direction). In some embodiments, the third segment 620 is configured to limit the sliding movement of the first jaw 631 of each cap-centering unit 603 relative to the first segment 611 and/or to limit the sliding movement of the second jaw 632 of each cap-centering unit 603 relative to the second segment 612 within a defined range or ranges.

For instance, in some embodiments, the first jaw 631 is formed with a first slot 635. The third segment 620 is formed with a first protrusion 621 that is smaller than the first slot 635 in the first decapper direction. When the first protrusion 621 is inserted into the first slot 635, there is a first space 637 between the first protrusion 621 and the first slot 635. This first space 637 defines the sliding movement range of the first jaw 631. Similarly, the second jaw 632 is formed with a second slot 636. The third segment 620 is formed with a second protrusion 622 that is smaller than the second slot 636 in the first decapper direction. When the second protrusion 622 is inserted into the second slot 636, there is a second space 638 between the second protrusion 622 and the second slot 636. This second space 638 defines the sliding movement range of the second jaw 632.

In some embodiments, the cap-holding unit 601 is connected to a head unit 604 and slidable relative to the head unit 604 in the first decapper direction. For instance, in some embodiments, the head unit 604 includes a first member 641, a rail 642 and a carriage 643. The first member 641 may include one or more return springs. The rail 642 is connected to the first member 641, and the carriage 643 is slidably coupled with the rail 642. The third segment 620 of the cap-holding unit 601 is connected to the carriage 643. This allows for the cap-holding unit 601 to move relative to the head unit 604 in the first decapper direction (e.g., moving left and right in FIG. 5E), enabling bulk adjustment in case any cap 512 deviates significantly from the central plane 614 of the channel 613. In some embodiments, the capping/decapping device 600 includes the head unit 604 (e.g., the head unit is an integral part of the capping/decapping device 600). In some embodiments, the head unit 604 is a component external to the capping/decapping device 600 (e.g., a component of the system 100).

In some embodiments, the cap-holding unit 601 is connected to a driving unit 605, which may be an integral part of the capping/decapping device 600, or a component external to the capping/decapping device 600 (e.g., a component of the system 100). The driving unit 605 is configured to move the cap-holding unit 601 and/or the head unit 604 in the third decapper direction (e.g., the z-direction in FIG. 5A, or up and down in FIG. 5E) that is substantially perpendicular to both of the first and second decapper directions. In some embodiments, the driving unit 605 includes a motor 651, a rail 652, a rail carriage 653, a first mounting member 654 and a second mounting member 655. The first mounting member 654 may be configured for mounting the cap-holding unit 601, the head unit 604 and/or the rail carriage 653. The first mounting member 654 may also include a lead screw nut bracket for coupling with the motor 651. The second mounting member 655 may be configured for mounting the rail 652, with the rail carriage 653 slidably coupled with the rail 652. The second mounting member 655 may be connected to other structures, such as the chassis and gantry unit 150 of the system 100. Through the first mounting member 654, the motor 651 is operable to move the cap-holding unit 601 and/or the head unit 604 along the rail 652 (e.g., in the third decapper direction). Because the tube body 511 of each sample tube 501 is held, for instance, by the tube holder 503, the movement of the cap-holding unit 601 in the third decapper direction will cause capping or decapping of each sample tube 501 that has been slid into the channel 613 of the cap-holding unit 601.

The capping/decapping device 600 may include one or more additional or optional components to facilitate the capping or decapping of a sample tube 501 in a controlled and/or safe manner. For instance, in some embodiments, the capping/decapping device 600 may include a switch flag 661 and a switch 662 to indicate the position of the cap-holding unit 601 and/or set a range for the movement of the cap-holding unit 601. The switch flag 661 may be connected to the first mounting member 654 and the switch 662 may be connected to the second mounting member 655, or vice versa.

### Exemplary Detection Unit

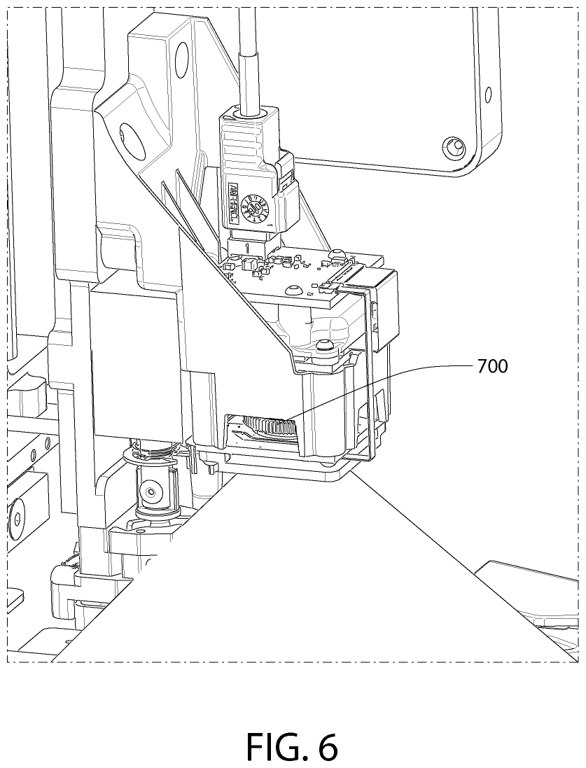

Referring to FIG. 6, there is shown an exemplary detection unit 700 in accordance with some exemplary embodiments of the present disclosure. The detection unit 700 (or one or more components of the detection unit) may be stationary, e.g., fixedly connected to the chassis and gantry unit 150 or any other stationary unit/module/device of the system 100. The detection unit 700 (or one or more components of the detection unit) may also be movable, e.g., movably connected to the chassis and gantry unit 150 or connected to any other movable unit/module/device of the system 100. For instance, in some embodiments, the detection unit 700 comprises one or more image cameras, and at least one camera is positioned at the gripper 200 and movable along with the gripper 200. This allows the camera to move inside the system 100 in all three axes and get to whichever position that is of interest. The camera may have a large field of view. In some embodiments, the detection unit 700 comprises multiple cameras, which may be placed at different locations in the system 100 or may be moved to different positions at the same time or at any time specified by the test. In some embodiments, the detection unit 700 includes one or more lights for illuminating the area of interest without lighting up the other parts of the system 100.

The detection unit 700 may be configured to perform a variety of functions. For instance, the detection unit 700 may be configured to detect microfluidic failures happening in an analytic module (e.g., the first analytic module 110, the second analytic module 120, or the third analytic module 130). The detection unit 700 may capture images of certain portions of the disc in the analytic module and analyze those images to determine if a certain failure has happened based on certain identified critical failure modes. In some embodiments, the detection unit 700 may be configured to detect microfluidic failures happening in each of the analytic modules. The detection unit 700 may also be configured to detect certain critical positions within the system 100, such as the critical positions for the chassis and gantry unit 150, the pipetting device 160, the gripper 200, the drawer 300 and/or other units/modules/devices. The detection of certain critical positions may be based on stationary fiducials placed in the system 100 and used as an auto calibration tool with minimal user input. The detection unit 700 may also be configured to perform certain assays. For instance, in some embodiments, a sample tube 501 contains a urine sample, and the consumables (e.g., the disc pack 301) include one or more urine strips. The urine sample may be placed on the one or more urine strips, for instance, by the pipetting device 160. The urine sample may change the color or cause the color shift on the one or more urine strips, which may be detected by the detection unit 700. In some embodiments, the detection unit 700 is configured for (i) detecting any microfluidic failure on the microfluidic disc in each of the plurality of analytical modules, (ii) detecting a position of the gripper, the holder, the rocker, the capping/decapping device, the drawer, or any combination thereof, thereby providing information for auto-calibration of the system, and (iii) performing one or more assays, or any combination thereof.

### Exemplary Thermal Management Unit

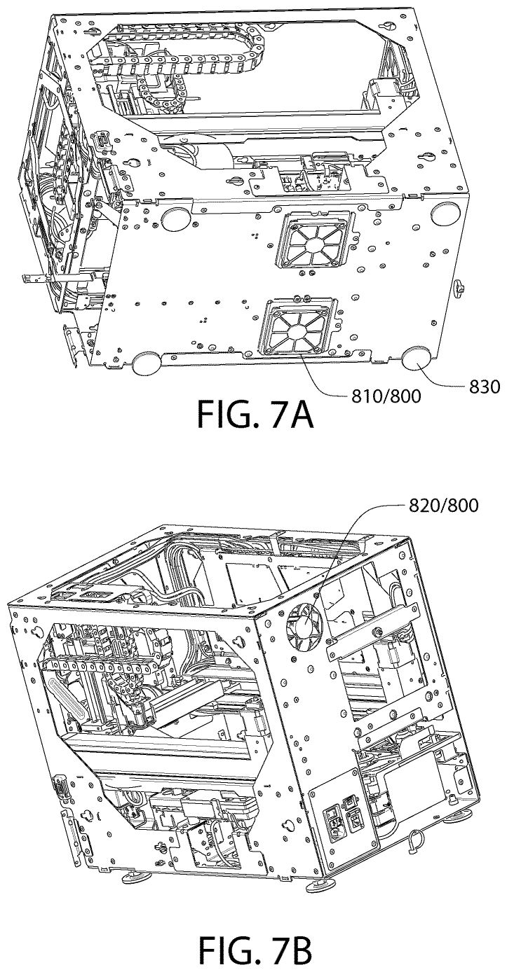

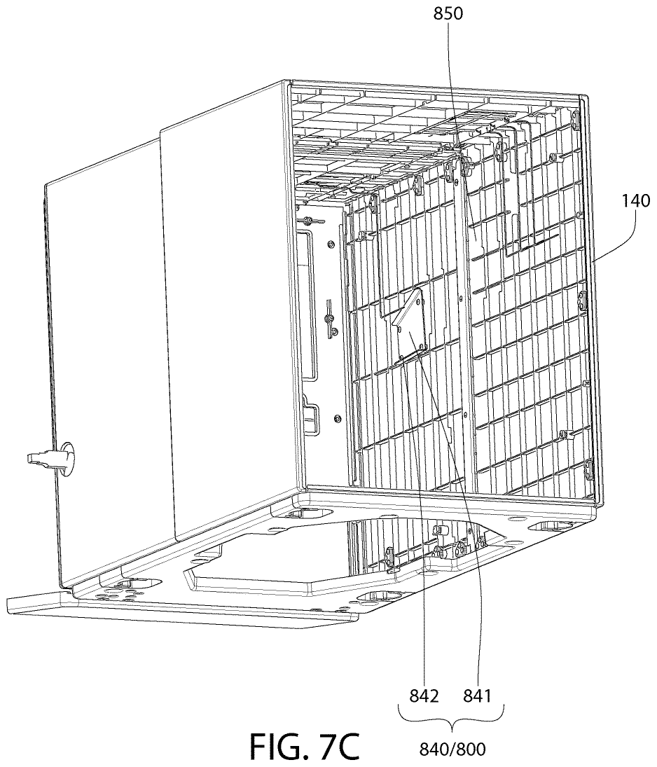

Referring to FIGS. 7A-7C, there is shown an exemplary thermal management unit 800 in accordance with some exemplary embodiments of the present disclosure. The thermal management unit 800 may be configured for maintaining the system 100 at a predetermined temperature range specified by the test. In some embodiments, the thermal management unit 800 includes one or more intake fans 810 and one or more exhaust fans 820. The one or more intake fans may be disposed at the bottom of the system 100. In some embodiments, one or more legs 830 are also disposed at the bottom of the system 100 to create a space for the one or more intake fans 810 to draw in fresh air. The one or more exhaust fans may be disposed on a side, such as a back side, of the system 100.

In some embodiments, the thermal management unit 800 includes one or more directing units 840, each configured for directing air flow to a certain region in the system 100. For instance, there may be a heat sink 850 in the system 100. The heat sink 850 may be a printed circuit board or a portion of it. A directing unit 840 may be configured to direct air flow to the heat sink 850 or surrounding area to effectively cool the heat sink 850. In some embodiments, the direction unit 840 includes a first member 841 and a pair of second members 842. The first member 841 may be in the form of a plate, skin or the like, and may be made of a material such as sheet metals or injected molded plastics. The first member 841 may be disposed in a way to create a gap between the first member 841 and the housing 140. The pair of second members 842 may be in the form of ribs or the like, forming a channel to direct air flow to the heat sink 850 or surrounding area. In some embodiments, the pair of second members 842 also serves as structural elements to support the first member 841.

Some embodiments or implementations are described with respect to the following clauses:

Clause A1. A gripper comprising:
a staging unit;
a plurality of fingers, wherein:
   fingers in the plurality of fingers are disposed circumferentially around a gripping axis;
   each respective finger in the plurality of fingers is pivotally connected to the staging unit at a middle portion thereof, thereby dividing each respective finger into a first finger portion and a second finger portion; and
   the second finger portion of each respective finger has a gripping part; and
a pushing unit connected to the staging unit and comprising a pushing member aligned with the gripping axis, wherein the pushing member is operably movable relative to the staging unit along the gripping axis to selectively abut outwardly against the first or second finger portion of each respective finger, thereby causing the gripping part of each respective finger to move inwardly or outwardly for gripping or releasing an object.

Clause A2. The gripper of clause A1, wherein:
the object is a disc comprising an aperture and a step along at least a portion of a perimeter of the aperture;
the gripping part of each respective finger is formed at an end of the second finger portion of each respective finger;
when moved inwardly, the gripping parts of the plurality of fingers are capable of passing through the aperture of the disc; and
when moved outwardly, the gripping parts of the plurality of fingers are engageable with the step of the disc to grip the disc.

Clause A3. The gripper of clause A2, wherein the staging unit is operably movable in one or more directions comprising a gripper direction that is substantially parallel to the gripping axis.

Clause A4. The gripper of clause A3, wherein the plurality of fingers comprises two, three, four, five, or more than five fingers.

Clause A5. The gripper of clause A4, wherein fingers in the plurality of fingers are substantially evenly spaced around the gripping axis.

Clause A6. The gripper of clause A5, wherein fingers in the plurality of fingers are substantially identical to each other.

Clause A7. The gripper of clause A6, wherein the gripping part of each respective finger comprises a hook facing outwardly.

Clause A8. The gripper of clause A7, wherein the pushing unit comprises a driving member to move the pushing member.

Clause A9. The gripper of clause A8, wherein the pushing member comprises:
a first pushing portion abutting outwardly against the first finger portion of each respective finger when the pushing member is at a first pushing position, thereby causing the gripping part of each respective finger to move inwardly; and
a second pushing portion abutting outwardly against the second finger portion of each respective finger when the pushing member is at a second pushing position, thereby causing the gripping part of each respective finger to move outwardly.

Claus A10. The gripper of clause A9, wherein the first or second pushing portion is substantially a truncated cone.

Clause A11. The gripper of clause A10, wherein bases of the first and second pushing portions face each other.

Clause A12. The gripper of clause A11, wherein the second pushing portion is smaller than the first pushing portion.

Clause A13. The gripper of clause A12, wherein the first and second pushing portions are spaced apart from each other along the gripping axis.

Clause A14. The gripper of clause A13, further comprising:
one or more posts connected to the staging unit and configured to abut against the object to help stabilize the object when the object is gripped by the plurality of fingers.

Clause A15. The gripper of clause A14, wherein:
the object is a disc comprising a disc surface substantially perpendicular to the gripping axis; and
the one or more posts comprise a plurality of datum posts disposed circumferentially around the gripping axis and radially outward of the plurality of fingers, each of the plurality of datum posts abutting against the disc surface in a direction substantially parallel to the gripping axis.

Clause A16. The gripper of clause A15, wherein the plurality of datum posts comprises two, three, four, five, or more than five datum posts.

Clause A17. A method for transporting a disc comprising an aperture and a step along at least a portion of a perimeter of the aperture, the method comprising:
providing the gripper of clause A1;
operating the pushing member to abut outwardly against the first finger portion of each respective finger, thereby causing the gripping part of each respective finger to move inwardly;
moving the staging unit to insert the gripping parts of the plurality of fingers into the aperture of the disc; and
operating the pushing member to abut outwardly against the second finger portion of each respective finger, thereby causing the gripping part of each respective finger to move outwardly and engage with the step of the disc.

Clause A18. The method of clause A17, further comprising:
moving the staging unit to lift up the disc.

Clause A19. The method of clause A18, further comprising:
moving the staging unit to transport the disc to a desired location.

Clause B1. A holder comprising:
a rack comprising one or more rack slots, wherein:
   each of the one or more rack slots is configured for housing a tube body of a sample tube in one or more sample tubes; and
   the rack is open at least partially at a first side of the rack to allow access to at least a portion of the tube body of the sample tube housed in any of the one or more rack slots;
a holder shaft disposed adjacent to the first side of the rack;
one or more retaining units, each associated with a corresponding rack slot in the one or more rack slots of the rack and comprising:
   a tube retaining member rotatably disposed at the holder shaft and comprising an edge at a side facing the first side of the rack; and
   a holder elastic member biasing the tube retaining member to push the edge against the tube body of the sample tube housed in the corresponding rack slot, thereby retaining the tube body of the sample tube in the corresponding rack slot of the rack.

Clause B2. The holder of claim B1, wherein:
the one or more rack slots comprise a plurality of rack slots; and
the one or more retaining units comprise a plurality of retaining units.

Clause B3. The holder of claim B2, wherein:
the plurality of rack slots comprises at least three rack slots; and
the plurality of retaining units comprises at least three retaining units.

Clause B4. The holder of clause B3, wherein the one or more sample tubes comprise:
a first sample tube containing a first type of blood;
a second sample tube containing a second type of blood;
a third sample tube containing urine; or
any combination thereof.

Clause B5. The holder of clause B4, wherein:
the first type of blood is ethylenediaminetetraacetic acid (EDTA) anticoagulated blood; and
the second type of blood is lithium heparin anticoagulated blood.

Clause B6. The holder of clause B5, wherein the tube retaining member of each of the plurality of retaining units is independently rotatable around the holder shaft.

Clause B7. The holder of clause B6, wherein each sample tube comprises a cap disposed outside of the corresponding rack slot of the rack.

Clause B8. The holder of clause B7, wherein:
the cap is coupled with the tube body by an interference fit;
the cap is color coded; or
both.

Clause B9. The holder of clause B8, wherein the edge of the tube retaining member is a wedge or a knife edge.

Clause B10. The holder of clause B9, wherein the holder elastic member is a coil spring having a first end connected to the rack or a stationary component and a second end connected to the tube retaining member.

Clause B11. The holder of clause B10, further comprising:
a first holder cam surface connected to the holder shaft and configured to engage with a first external component to rotate the holder, thereby disengaging the edge of the tube retaining member of each of the one or more retaining units from the sample tube inserted in each of the one or more rack slots of the rack.

Clause B12. The holder of clause B10, wherein:
the holder elastic member pushes the tube retaining member toward the first side of the rack, thereby engaging the edge of the tube retaining member with the sample tube inserted in the corresponding rack slot.

Clause C1. a rocker comprising:
a rocker mounting unit;
a rocker shaft having a rotational axis and rotatably mounted on the rocker mounting unit, wherein the rocker shaft is configured for fixedly connecting a holder containing one or more sample tubes;
a rocker driving unit connected to the rocker shaft and configured to rotate the rocker shaft clockwise and counter-clockwise around its rotation axis between a positive angle and a negative angle, thereby rocking the holder containing one or more sample tubes.

Clause C2. The rocker of clause C1, wherein the holder is the holder of clause B1.

Clause C3. The rocker of clause C2, wherein:
the rocker mounting unit comprises a first mounting member and a second mounting member spaced apart in a direction substantially parallel to the rotation axis;
the rocker shaft is rotatably connected to the first mounting member; and
the holder is disposed between the first and second mounting members.

Clause C4. The rocker of clause C3, wherein the rocker driving unit comprises:
a rocker motor; and
a coupler selectively connecting the rocker shaft with the holder.

Clause C5. The rocker of clause C4, wherein the rocker motor is a stepper motor.

Clause C6. The rocker of clause C4, wherein the rocker driving unit further comprises:
a lock operable to selectively engage with the coupler to prevent undesirable rotation of the holder.

Clause C7. The rocker of clause C6, wherein:
the coupler comprises a notch; and
the lock comprises:
   a plugging portion insertable into the notch of the coupler; and
   a cam surface configured to engage with an external component to push the plugging portion into the notch of the coupler or pull the plugging portion out of the notch.

Clause C8. The rocker of clause C7, wherein the lock is activated to engage with the coupler in response to a detection of a failure.

Clause C9. The rocker of clause C8, wherein the lock is activated by a spring.

Clause C10. The rocker of clause C8, wherein the failure comprises:
a tilting of the holder beyond a predetermined angle.

Clause C11. The rocker of clause C1, further comprising:
a rocker sensing unit configured to detect a position of the one or more sample tubes contained in the holder.

Clause C12. The rocker of clause C11, wherein the rocker sensing unit is configured to further determine if any of the one or more sample tubes is positioned improperly.

Clause C13. The rocker of clause C12, wherein the rocker sensing unit comprises one or more sensors disposed below the rocker shaft.

Clause C14. The rocker of any one of clauses C1-C13, wherein the rocker driving unit is configured to rotate the rocker shaft clockwise and counter-clockwise around its rotation axis rocker at a controlled speed, a controlled acceleration, a controlled deceleration, a controlled frequency, a controlled number of times, or any combination thereof.

Clause D1. A capping/decapping device comprising:
a cap-holding unit comprising:
   a first segment;
   a second segment spaced apart from the first segment in a first decapper direction;
   a channel between the first and second segments and configured to allow one or more caps of one or more sample tubes contained in a holder to slide into the cap-holding unit in a second decapper direction that is substantially perpendicular to the first decapper direction;
   a first shoulder at a side of the first segment facing the channel and configured to allow a first portion of a lower side of the one or more caps to sit on the first shoulder; and
   a second shoulder at a side of the second segment facing the channel and configured to allow a second portion of the lower side of the one or more caps to sit on the second shoulder;
one or more cap-centering units, each comprising:
   a first jaw disposed at the first segment of the cap-holding unit above the first shoulder and slidable relative to the first segment in the first decapper direction;
   a first centering elastic member biasing the first jaw in the first decapper direction towards a central plane of the channel;
   a second jaw disposed at the second segment of the cap-holding unit above the second shoulder and slidable relative to the second segment in the first decapper direction; and
   a second centering elastic member biasing the second jaw in the first decapper direction towards the central plane of the channel,
   thereby centering a cap, which has been slid into the channel, at a position in accordance with the central plane of the channel.

Clause D2. The capping/decapping device of clause D1, wherein the cap-holding unit is connected to a head unit and slidable relative to the head unit in the first decapper direction.

Clause D3. The capping/decapping device of clause D2, wherein the head unit is a component of the capping/decapping device.

Clause D4. The capping/decapping device of clause D2 or D3, wherein the head unit is a component external to the capping/decapping device.

Clause D5. The capping/decapping device of any one of clauses D1-D4, wherein:
each of the first and second shoulders comprises one or more indents; and
each of the one or more indents of the first or second shoulders is aligned with a cap-centering unit in the first decapper direction and configured for a cap in the one or more caps to sit.

Clause D6. The capping/decapping device of any one of clauses D1-D5, wherein the first and second shoulders abut against the lower side of the one or more caps in a third decapper direction that is substantially perpendicular to both of the first and second decapper directions, thereby facilitating capping or decapping of the one or more sample tubes.

Clause D7. The capping/decapping device of any one of clauses D1-D6, wherein the first and second shoulders hold any cap once it is decapped from a sample tube in the one or more sample tubes.

Clause D8. The capping/decapping device of any one of clauses D1-D7, wherein the cap-holding unit further comprises:
a third segment formed or coupled with the first and second segments and configured to limit a sliding movement range of the first or second jaw of each of the one or more cap-centering units relative to the first or second segment.

Clause D9. The capping/decapping device of any one of clauses D1-D8, wherein the one or more cap-centering units comprise:
a plurality of cap-centering units disposed along the second decapper direction.

Clause D10. The capping/decapping device of clause D9, wherein the plurality of cap-centering units comprises:
two, three, four, five or more than five cap-centering units.

Clause D11. The capping/decapping device of any one of clauses D1-D10, wherein for each of the one or more cap-centering units:
the first centering elastic member is fixedly connected to the first segment; and
the second centering elastic member is fixedly connected to the second segment.

Clause D12. The capping/decapping device of any one of clauses D1-D11, wherein for each of the one or more cap-centering units:
the first or second centering elastic member is a leaf spring.

Clause D13. The capping/decapping device of any one of clauses D1-D12, further comprising:
a driving unit connected to the cap-holding unit and configured to move the cap-holding unit in a third decapper direction that is substantially perpendicular to both of the first and second decapper directions, thereby capping or decapping each of the one or more sample tubes that has been slid into the channel of the cap-holding unit.

Clause E1. a drawer comprising:
one or more drawer nests, each configured for housing a pack comprising a casing and one or more items in the casing, wherein each respective drawer nest in the one or more drawer nests comprises:
   a nest slot at a front side of the drawer to allow insertion of a corresponding pack into the respective drawer nest;
   a nest opening at an upper side of the drawer to allow access to the one or more items contained in the corresponding pack; and
   a nest frame above the corresponding pack to prevent the casing of the corresponding pack from moving upwardly.

Clause E2. The drawer of clause E1, wherein each respective drawer nest in the one or more drawer nests further comprises:
a nest position sensor to detect a position of the corresponding pack in the respective drawer nest; and
an indicator to indicate, according to the detection by the nest position sensor, whether the corresponding pack is properly positioned in the respective drawer nest.

Clause E3. The drawer of clause E2, wherein the indicator indicates whether the corresponding pack is properly positioned in the respective drawer nest visually, aurally, or both visually and aurally.

Clause E4. The drawer of clause E3, wherein the indicator indicates whether the corresponding pack is properly positioned in the respective drawer nest by color.

Clause E5. The drawer of any one of clauses E2-E4, wherein the indicator is disposed at a front or upper side of the respective drawer nest.

Clause E6. The drawer of any one of clauses E2-E5, wherein the one or more drawer nests comprises:
a first drawer nest configured for housing a first pack comprising one or more microfluidic discs, wherein the nest opening of the first drawer nest is substantially circular; and
a second drawer nest configured for housing a second pack comprising one or more reagent wells and one or more pipetting tips.

Clause E7. The drawer of clause E6, wherein the one or more microfluidic discs comprises:
a first microfluidic disc configured for a complete blood count test;
a second microfluidic disc for an immunoassay;
a third microfluidic disc for a clinical chemistry analysis; or
any combination thereof.

Clause E8. The drawer of clause E6 or E7, wherein the first and second drawer nests are formed as a unitary piece.

Clause E9. The drawer of any one of clauses E6-E8, wherein the first drawer nest further comprises:
a nest base having a nest retaining member configured to engage with a retaining member of the casing of the corresponding pack to restrict rotation of the casing of the corresponding pack relative to the respective drawer nest.

Clause E10. The drawer of clause E9, wherein the first drawer nest further comprises:
a nest camera disposed at the nest base and configured to retrieve information from a barcode on the casing of the corresponding pack.

Clause E11. The drawer of any one of clauses E1-E10, further comprising:
a drawer chassis configured for coupling with a rail in a housing adjacent to a door, wherein the one or more drawer nests are mounted on the drawer chassis; and
a drawer roller connected to the drawer chassis and configured for opening the door.

Clause E12. The drawer of clause E11, wherein:
the door is connected to the housing by one or more spring hinges and is rotatable relative to the housing around a door hinge axis;
the drawer chassis is slidable in a direction substantially perpendicular to the door hinge axis;
the drawer roller is connected to the drawer chassis such that it is able to move along with the drawer chassis and rotatable around a roller rotational axis that is substantially parallel to the door hinge axis;
as the drawer chassis slides toward the door, the drawer roller rolls on an inner surface of the door, thereby pushing the door to open; and
as the drawer chassis slides away from the door, the one or more spring hinges pull and close the door.

Clause F1. A system comprising:
the gripper of any one of clauses A1-A16;
the holder of any one of clauses B1-B11;
the rocker of any one of clauses C1-C14;
the capping/decapping device of any one of clauses D1-D13;
the drawer of any one of clauses E1-E12;
a pipetting device;
a plurality of analytical modules, each configured for receiving a microfluidic disc and analyzing a biological sample;
a detection unit configured for one or more of:
   (i) detecting any microfluidic failure on the microfluidic disc in each of the plurality of analytical modules;
   (ii) detecting a position of the gripper, the holder, the rocker, the capping/decapping device, the drawer, or any combination thereof, thereby providing information for autocalibration of the system; and
   (iii) Performing One or More Assays;
a thermal management unit configured for maintaining the system at a predetermined temperature range; or
any combination thereof.

Clause F2. The system of clause F1, wherein the plurality of analytical modules comprises:
a first analytical module configured for receiving a first microfluidic disc and performing a complete blood-count test;
a second analytical module configured for receiving a second microfluidic disc and performing an immunoassay; and
a third analytical module configured for receiving a third microfluidic disc and performing a clinical chemistry analysis.

Clause F3. The system of clauses F1 or F2, wherein the detection unit comprises one or more image cameras.

Clause F4. The system of any one of clauses F1-F3, further comprising:
a housing comprising a door and a user interface;
a chassis; and
a gantry.

Clause F5. The system of any one of clauses F1-F4, further comprising:
a controlling unit for operating the gripper, the holder, the rocker, the capping/decapping device, the drawer, the pipetting device, the plurality of analytical modules, the detection unit, the thermal management unit, or any combination thereof.

Clause F6. A method for determining one or more analytes in a biological sample using the system of any one of clauses F1-F5.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will be understood that the terms “left” or “right”, “top” or “bottom”, “lower” or “upper”, “interior” or “exterior”, “inward” or “outward” and etc. are used to describe features of the exemplary embodiments with reference to the positions of such features as displayed in the figures. It will be understood that, although the terms “first,” “second,” etc. may be used herein to describe various elements, these elements should not be limited by these terms. These terms are only used to distinguish one element from another. For example, a first element could be termed a second element, and, similarly, a second element could be termed a first element, without changing the meaning of the description, so long as the “first element” and the “second element” are renamed consistently.

As used herein, the term “and/or” as used herein refers to and encompasses any and all possible combinations of one or more of the associated listed items. It will be further understood that the terms “include”, “includes”, “including”, “comprise”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein in conjunction with a numeral, refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

The term “if” used herein is, optionally, construed to mean “when” or “upon” or “in response to determining” or “in response to detecting” or “in accordance with a determination that,” depending on the context. Similarly, the phrase “if it is determined” or “if [a stated condition or event] is detected” used herein is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

When a reference number is given an “i^{th}” denotation, the reference number refers to a generic component, set, or embodiment. For instance, a “unit i” refers to the i^{th} unit in a plurality of units.

All references cited herein are incorporated herein by reference in their entirety and for all purposes to the same extent as if each individual publication or patent or patent application was specifically and individually indicated to be incorporated by reference in its entirety for all purposes.

1. A system comprising:
one or more analytical modules, each configured for receiving a corresponding microfluidic disc and performing optical analysis of a corresponding biological sample,
wherein the one or more analytical modules comprise a plurality of analytical modules, the plurality of analytical modules capable of performing a full suite of tests, and
wherein the one or more analytical modules comprise:
a first analytical module configured for receiving a first microfluidic disc and measuring one or more hematological parameters;
a second analytical module configured for receiving a second microfluidic disc and performing an immunoassay;
a third analytical module configured for receiving a third microfluidic disc and performing a clinical chemistry analysis; or
any combination thereof.

2-3. (canceled)

4. The system of claim 1, wherein analytical modules in the plurality of analytical modules are capable of performing optical analyses independently of each other or concurrently with each other.

5-6. (canceled)

7. The system of claim 1, further comprising:
a gripper configured for transporting the corresponding microfluidic disc to or from each of the one or more analytical modules;
a holder configured for holding one or more sample tubes;
a rocker configured for mixing or homogenizing one or more biological samples in the one or more sample tubes;
a capping/decapping device configured for decapping and/or recapping a cap from a sample tube in the one or more sample tubes;
a drawer configured for receiving consumables;
a pipetting device configured for pipetting the corresponding biological sample to the corresponding microfluidic disc;
a detection unit configured for one or more of:
(i) detecting any microfluidic failure on the microfluidic disc in each of the plurality of analytical modules;
(ii) detecting a position of the gripper, the holder, the rocker, the capping/decapping device, the drawer, or any combination thereof, thereby providing information for auto-calibration of the system; and
(iii) performing one or more assays;
a thermal management unit configured for maintaining the system at a predetermined temperature range; or
any combination thereof.

8. The system of claim 7, wherein the detection unit comprises one or more image cameras.

9. The system of claim 8 further comprising:
a housing comprising a door and a user interface;
a chassis; and
a gantry.

10. The system of claim 9 further comprising:
a controlling unit for operating the gripper, the holder, the rocker, the capping/decapping device, the drawer, the pipetting device, the plurality of analytical modules, the detection unit, the thermal management unit, or any combination thereof.

11. The system of claim 10, wherein the one or more analytical modules comprise at least three analytical modules powered by a single and unified architecture and capable of being performed in a modular and streamlined platform.

12. The system of claim 11, wherein the corresponding biological sample is aliquoted into the corresponding microfluidic disc for the optical analysis.

13. The system of claim 12, wherein the system is configured to accept a single consumable kit per sample.

14. The system of claim 13, wherein the one or more analytical modules are capable of providing a plurality of biomarkers results.

15. The system of claim 14, wherein the one or more analytical modules are capable of providing 2, 3, 4, 5, 6, 7, 8, 9, 10, 11, 12, 13, 14, 15, 16, 17, 18, 19, 20, 30, 40, 50, 60, 70, 80, 90, 100 or more than 100 biomarkers results.

16. The system of claim 14, wherein the one or more analytical modules are capable of providing more than 5 biomarkers results.

17. The system of claim 16, wherein optical analysis of the corresponding biological sample is performed using a single workflow with user-selectable reporting.

18. The system of claim 17, wherein the system is automated.

19. The system of claim 18, wherein the system is configured to accept one or more sample tubes, homogenize one or more biological samples contained in the one or more sample tubes, and analyze the homogenized one or more biological samples without any user intervention.

20. The system of claim 19, wherein the system is configured to assess a quality of samples, reagents, and/or protocols.

21. The system of claim 19, wherein the system is configured to transmit information about a quality of samples, reagents, and/or protocols using telemetry information.
